(19) United States
(12) Patent Application Publication
(10) Pub. No.: US 2006/0200198 A1
(43) Pub. Date: Sep. 7, 2006

(54) Systems and methods for closing a tissue opening

(76) Inventors: Daniel J. Riskin, Los Angeles, CA (US); Andrew D. Fox, Westborough, MA (US); James D. Mills, Wilmington, MA (US)

Correspondence Address: WILMER CUTLER PICKERING HALE AND DORR LLP, 60 STATE STREET, BOSTON, MA, 02109, US

(21) Appl. No.: 11/217,127

(22) Filed: Aug. 31, 2005

Related U.S. Application Data
(60) Provisional application No. 60/522,207, filed on Aug. 31, 2004.
(60) Provisional application No. 60/593,236, filed on Dec. 26, 2004.
(60) Provisional application No. 60/594,771, filed on May 5, 2005.

(51) Int. Cl. A61B 17/08

(52) U.S. Cl. 606/215

(57) ABSTRACT

The present invention is directed to tissue closure devices for closing a tissue opening, including one or more closure components, each closure component having a first member and a second member, each of the first and second members having a first surface that adheres to a tissue surface proximate to the tissue opening, each of the first and second members having a second surface substantially orthogonal to the first surface, and each of the first and second members having a transitional region between the first surface and the second surface which is contoured to evert an edge of the tissue opening upon the drawing together of the first and second members. The present invention is also directed to systems and kits, dressing systems, and methods for tissue repair and closure.

### CROSS-REFERENCE TO RELATED APPLICATIONS

This application claims the benefit of U.S. Provisional Patent Application Ser. No. 60/522,207 filed Aug. 31, 2004; U.S. Provisional Patent Application Ser. No. 60/593,236 filed Dec. 26, 2004; and U.S. Provisional Patent Application Ser. No. 60/594,771 filed May 5, 2005; the entire contents of all three provisional applications are incorporated herein by reference.

### TECHNICAL FIELD

This invention relates generally to tissue closure devices, systems and kits, dressing systems, and methods for tissue repair and closure.

### BACKGROUND

Closure of tissue openings, such as, for example, for surgical incisions and accidental lacerations or wounds, is critical both to minimize the risk of infection and to promote optimal healing of the wound or incision. Both of these outcomes require rapid wound closure and careful skin edge approximation. Closing a tissue opening or wound requires a mechanism for drawing both sides of a tissue opening together to promote healing and to reduce the formation of scar tissue.

Previous wound closure systems included various categories of materials passed through the skin, such as staples and sutures, substances that cover skin edges and hold them adjacent, such as glues, and adherent structures, such as strips. Common methods for closing tissue openings caused by lacerations or surgical incisions are suturing and stapling. Both of these procedures are invasive, which can traumatize and compromise the integrity of the tissue opening and the nutrient blood supply to the healing tissue edges. They cause pain, increase the possibility of infection, expose the surgeon, as well as the patient, to blood-born disease, leave behind scars, and require a follow-up visit for suture or staple removal. Surgical glue is also used, but has only been proven adequate for small wounds where skin edges are not widely separated or under tension during closure.

Surgeons have become skilled in the various techniques of suturing to minimize the resulting blemish that occurs during the healing process. These methods require a threshold of dexterity that many care providers do not possess. This is particularly true in emergency situations, which often require immediate treatment to secure the tissue opening to allow for transport or until such time as proper surgery is possible. Suturing, even by a skilled surgeon, punctures and stresses tissue causing scarring. A sutureless tissue opening closure system would be a great benefit in many situations.

Adhesive tissue closures have been introduced that can effectively close some types of tissue openings without inflicting the additional injury inherent in suturing. Adhesive closures have a backing to provide solid structure, and have an adhesive layer for adhering to the skin.

An exemplary early attempt in non-invasive wound closures used a pair of strips of fabric having adhesive backing. The strips of fabric were applied in parallel on either side of the tissue opening and were constructed with threads extending transversely to bridge the tissue opening. A compressive force was applied across the tissue opening by tying opposing ends of the transverse threads of adjacent strips.

In another device, the distal ends of the bridging threads of one adhesive strip were interconnected by another pulling strip, allowing the bridging threads to be manipulated in concert. This configuration required that the bridging threads or filaments of each of the adhesive strips be interlaced to enable the pulling strips to be pulled across the tissue opening and secured.

However, as with many prior systems, the manipulation of a loose assembly of multiple parts in an emergency and possibly life-threatening situation is a challenging undertaking.

In addition, some adhesives have been used that have utility for skin contacting applications, provide good skin compatibility, and are hydrophobic, so that they tend not to remain in the interior of tissue openings. However, their tensile strength is only sufficient for some uses, for example, for closing or sealing skin cracks, not for holding major tissue openings closed against the range of motion to which the skin or any tissue is normally subjected.

In addition to the typical wound closure devices, referred to also as devices to close a tissue opening herein, other aspects of wound closure and care have deficiencies. For example, kits for wound irrigation and closure do not allow the introduction of sterile or clean fluids before beginning a procedure. They also do not allow for different sections to be cleaned or sterilized for different parts of the procedure. Post procedural care is critical to achieve optimal healing results, but is often not properly attended to in order to minimize scarring and optimize wound healing. Currently available wound dressings are not specialized for different time periods within the healing cycle. By ignoring the changing physiology and needs of the healing wound, current dressings do not provide an optimal healing environment.

There is still a need to simplify and improve upon devices and methods for closing tissue openings and their application and provide for wound care in general.

### SUMMARY OF THE INVENTION

The systems and methods for closing surgical incisions and non-surgical wounds of various embodiments of the invention provide for improved wound care.

One aspect of the invention provides for a closure device for closing a tissue opening, including one or more closure components, each closure component having a first member and a second member, each of the first and second members having a first surface that adheres to a tissue surface proximate to the tissue opening, the first surface having at least one adhesive, each of the first and second members having a second surface substantially orthogonal to the first surface and having at least one connective element on the second surface, and each of the first and second members having a transitional region between the first surface and the second surface which is contoured to evert an edge of the tissue opening upon the drawing together of the first and second members by the engagement of at least one connective element.

In some embodiments, the connective element is a locking mechanism. In various embodiments, the locking mechanism includes a locking member extending from the second surface of the first member and the second surface of the second member having a receiving member. In one embodiment, each of the second surfaces has at least one locking member and at least one receiving member, such that in some embodiments, the first member and the second member are identical. In one embodiment, the connective elements include a ball and socket mechanism. In another embodiment, the connective elements include a rachet mechanism. In yet another embodiment, the connective elements include a suture ligature, and in another embodiment, the connective elements have a lock and key mechanism. In some embodiments, the connective elements include magnets. In additional embodiments, the connective elements have a photobonded mechanism. In one embodiment, the connective elements include a staple. In some embodiments, the connective element of the first member engages the connective element of the second member when the second surface of the first member is in operative relation to the second surface of the second member. In various embodiments, the engaged connective elements provide a vertical force orthogonal to a plane of the tissue opening on at least one edge of the tissue opening. In further embodiments, the process of engaging the connective elements brings edges of the tissue opening together and everts the edges of the tissue opening. In one embodiment, the connective element is an adhesive, which in some embodiments is glue.

In some embodiments, the first and second members are releasably coupled. In some embodiments, the closure device further includes a closure release mechanism. In one embodiment, the closure release mechanism is a push button release hole.

In some embodiments, the closure device further includes a mechanism to adjust the placement of the members. In various embodiments, the mechanism to adjust the placement of the members is a sliding track. In additional embodiments, the mechanism to adjust the placement of the members is the adhesive on the first surface providing for repositioning of the member. In further embodiments, the mechanism to adjust the placement of the members is a hinge. In some embodiments, the mechanism to adjust the placement of the members is a ball bearing. Furthermore, in some embodiments, the mechanism to adjust the placement of the members includes an element to secure its position and prevent or at least minimize further unintended adjustment. In various embodiments, the securing element is mechanical, and in some embodiments the securing element is an adhesive.

In various embodiments, the closure device further includes a sliding lock to position and hold the first and second members in proximity to each other. In some embodiments, the closure device further has protrusions extending from the member to facilitate handling of the components. In additional embodiments, the closure device further includes an elastomeric base between the adhesive and the first surface of at least one member.

In some embodiments, the members, or a portion of the members, are plastic. In other embodiments, the members, or a portion of the members, are metal. In some embodiments the members are bioabsorbable. Furthermore, in some embodiments, at least one of the members is flexible.

In additional embodiments, the closure device further includes an agent to detect at least one of the presence of an infection or the presence of an impending infection. In some embodiments, the agent is present in a covering placed over the tissue opening. In various embodiments, the agent is present in at least one member.

In some embodiments, the adhesive is a semi-permanent skin bonding agent. In various embodiments, the semi-permanent skin bonding agent is a pressure sensitive adhesive. In other embodiments, the semi-permanent skin bonding agent is a skin glue. Further, in some embodiments, the closure device further comprises a second adhesive, such as for example, cyanoacrylate, or other biological glue, or a pressure sensitive adhesive. A non-limiting example of a biological glue is a thrombin gel. In some embodiments, at least one member comprises an opening for application of a second adhesive provided in a plane that is substantially parallel to the first surface and spaced apart from the first surface, the opening extending through to the first surface, and in some embodiments the opening is for application of a second adhesive. In some embodiments, the opening does not extend through the first adhesive.

In some embodiments, each member comprises two or more layers. In further embodiments, at least one layer is porous. In additional such embodiments, the porous layer is sealed by a material that provides pressure-induced porosity. In some embodiments, the second adhesive is present in the porous layer.

In various embodiments of the invention, the transitional region is tapered. In some embodiments, the transitional region is beveled. In other embodiments, the transitional region is arcuate. In further embodiments, the transitional region is chamfered, and in yet further embodiments, the transitional region is sloped.

Another aspect of the invention provides for a kit for a wound closure system, including a container having, a first component or layer of materials for cleaning a tissue opening; and a second component including a closure device, wherein at least two of the components of the kit are physically separated.

In some embodiments, the materials for cleaning the tissue opening include an irrigation system. In various embodiments, the irrigation system includes a container of saline and a non-splash syringe.

In some embodiments, the closure device includes one or more closure components, each closure component having a first member and a second member; each of the first and second members having a first surface that adheres to a tissue surface proximate to the tissue opening, the first surface having at least one adhesive; each of the first and second members having a second surface substantially orthogonal to the first surface and having at least one connective element on the second surface; and each of the first and second members having a transitional region between the first surface and the second surface which is contoured to evert an edge of the tissue opening upon the drawing together of the first and second members by engaging the connective elements. In some embodiments, each member of the closure device has two or more layers. In additional embodiments, at least one layer is porous. In some embodiments, the porous layer is sealed by a material that provides pressure-induced porosity. In further embodiments, the second adhesive is present in the porous layer. In additional embodiments, the closure device includes suturing materials.

In various embodiments, the kit further includes a component having instructional materials. In further embodiments, the kit further includes a sterile field component. In some such embodiments, the sterile field component includes sterile gloves, and in further such embodiments, the sterile field component includes a sterile field to allow preparation for a sterile tissue closure procedure. In some embodiments, the components subsequent to the sterile field component are sterile.

In additional embodiments, the kit further includes materials for application of an adhesive. In some embodiments, the materials for application of an adhesive include at least one of an applicator tip, an adhesive, and an adhesive dispenser. In some embodiments, the adhesive is cyanoacrylate.

In some embodiments, the container is a sealed tray. In additional embodiments, at least a portion of the container is covered by a removable barrier. In various embodiments, removing the removable barrier provides a window to a specific component of the kit. In further embodiments, the kit further has an external cover. In some such embodiments, the container defines an opening for access to at least a portion of the container, which opening is covered by the external cover. In various embodiments, a fluid can be introduced into at least a portion of the container through the opening. Furthermore, in some embodiments, the container comprises a receptacle for receiving the fluid.

According to another aspect, the invention provides for a dressing system to facilitate healing of a tissue opening, including one or more bandages for dressing a tissue opening, the bandages being appropriate for specific time periods of healing of a tissue opening, and at least one of the bandages having an additive to facilitate healing of a tissue opening.

In some embodiments, the dressing system further includes instructional material or a manual. In various embodiments the instructional material includes information regarding placement of one or more bandages at one or more specified time periods. In some embodiments, the time periods correspond to phases of healing.

In additional embodiments, at least one bandage prevents or at least minimizes the penetration of moisture to the tissue opening. In further embodiments, at least one bandage has an agent for pain relief. In some embodiments, at least one bandage includes an agent to detect at least one of the presence of an infection and the presence of an impending infection. In one embodiment, at least one bandage has an indicator responsive to changes associated with infection. In one such embodiment, the indicator reacts to at least one of a specific pH level or protein exudates.

In further embodiments, at least one bandage is transparent. In additional embodiments, at least one bandage has at least one agent to facilitate healing. In some embodiments, the agent to facilitate healing is Vitamin E. In yet further embodiments, at least one bandage includes agents to maximize scar softness and reduce discoloration. In some embodiments, at least one bandage has a solvent for removal of a closure device. In one embodiment, the dressing system has at least one bandage that prevents penetration of moisture to the tissue opening and at least one bandage comprising Vitamin E.

In some embodiments, the dressing system further includes a receptacle having at least one agent for application to the tissue opening or to at least one bandage.

Another aspect of the invention provides for a dual-adhesive closure device for closing a tissue opening, having one or more closure components, each closure component having a first member and a second member, each of the first and second members having a first surface that adheres to a tissue surface proximate to the tissue opening, the first surface having at least one adhesive, each of the first and second members having a second surface substantially orthogonal to the first surface and having at least one connective element on the second surface, each of the first and second members having a transitional region between the first surface and the second surface which is contoured to evert an edge of the tissue opening upon the drawing together of the first and second members by the engagement of the connective elements, and a second adhesive applied to secure the first surface to tissue approximately adjacent to at least one edge of the tissue opening.

In some embodiments, both members include a first layer, a second layer, and a reservoir between the first layer and the second layer, the reservoir containing the second adhesive. In various embodiments, the second adhesive is released when the connective elements are engaged. In additional embodiments, the second adhesive is applied manually. In further embodiments, the adhesive is released from a reservoir within the member. In some embodiments, the adhesive is applied from an external source and spreads between the first surface and an adjacent tissue surface. In various embodiments, the second adhesive is cyanoacrylate.

In some embodiments, each member of the dual-adhesive closure device comprises two or more layers. In additional embodiments, at least one layer is porous. In further embodiments, the porous layer is sealed by a material that provides pressure-induced porosity. In some embodiments, the second adhesive is present in the porous layer.

An additional aspect of the invention provides for a closure system for a tissue opening, including a temporary closure device comprising a deformable perimeter defining an opening, the deformable perimeter having a first surface and at least one adhesive on the first surface.

In some embodiments, the temporary closure device surrounds the tissue opening. In additional embodiments, the temporary closure device is oval. In various embodiments, the adhesive is present on two spaced sections of the first surface. In some embodiments, the adhesive is a pressure sensitive adhesive.

In various embodiments, the temporary closure device, or part thereof, is plastic. In additional embodiments, the temporary closure device includes sections of material forming the perimeter, the perimeter having at least one material in each section, the perimeter further having nonidentical sets of materials used in at least two sections, and wherein the sections with nonidentical sets of materials are not adjacent.

In further embodiments, the temporary closure device is sufficiently compliant to deform and exert pressure on opposing sides of a tissue opening and is sufficiently rigid to maintain the deformed shape and maintain the pressure on opposing sides of the tissue opening. In some embodiments, opposing sides of the perimeter are pressed together, exerting a force on tissue underneath the temporary closure device to bring edges of a tissue opening closer together.

In some embodiments, the closure system further includes a closure device, having one or more closure components, each closure component including a first member and a second member, each of the first and second members having a first surface that adheres to a tissue surface proximate to the tissue opening, the first surface having at least one adhesive, each of the first and second members having a second surface substantially orthogonal to the first surface and having at least one connective element on the second surface, and each of the first and second members having a transitional region between the first surface and the second surface which is contoured to evert an edge of the tissue opening upon the drawing together of the first and second members by the engagement of the connective elements.

In some embodiments, each member of the closure device has two or more layers. In additional embodiments, at least one layer is porous. In further embodiments, the porous layer is sealed by a material that provides pressure-induced porosity. In some embodiments, a second adhesive is present in the porous layer.

In various embodiments, the closure system further includes an applicator device for applying the closure components, the applicator device comprising at least one of a mechanism to hold the closure components, a mechanism to release the closure components, and a mechanism to affix the closure components. In some embodiments, the applicator device has a handle, a plunger, and/or a reservoir. In additional embodiments, the reservoir holds an adhesive. In some embodiments, the mechanism to affix the closure components releases an adhesive. In additional embodiments, the applicator device further comprises a light-based guide to assist with the placement of the members. In additional embodiments, the applicator device further includes a mechanism to adjust placement of the closure components. In still additional embodiments, the applicator device further includes a mechanism for lowering the closure components onto the tissue surface. In some embodiments, the applicator device further includes a mechanism for securing the closure components. In additional such embodiments, the mechanism for securing the closure components comprises an adhesive released from the applicator device. In various embodiments, the adhesive is cyanoacrylate.

Another aspect of the invention provides for a method for closing a tissue opening, including positioning a closure device for closing a tissue opening, the closure device having one or more closure components, each closure component having a first member and a second member; each of the first and second members having a first surface that adheres to a tissue surface proximate to the tissue opening, the first surface having at least one adhesive, each of the first and second members having a second surface substantially orthogonal to the first surface and having at least one connective element on the second surface, and each of the first and second members having a transitional region between the first surface and the second surface which is contoured to evert an edge of the tissue opening upon the drawing together of the first and second members by the engagement of the connective elements; drawing the first and second members into proximity such that the connective element on the second surface of the first member engages the connective element on the second surface of the second member; and securing each of the first and second members to a tissue surface.

In some embodiments of the invention, the closure members are positioned adjacent to an edge of the tissue opening. In additional embodiments, the transitional region is tapered. In further embodiments, edges of the tissue opening adhere to the transitional region and are everted. In some embodiments, bringing the first and second members into proximity such that the connective element on the second surface of the first member engages the connective element on the second surface of the second member everts the wound edge. In one embodiment, the transitional region is beveled and drawing the first and second members into proximity such that the connective element on the second surface of the first member engages the connective element on the second surface of the second member brings the beveled transition region of the first member into proximity with the beveled transition region of the second member and everts the wound edge.

Additional embodiments of the method further include adjusting the position of at least one of the closure members after positioning it. In some embodiments, the first and second members are secured before bringing them into proximity. In other embodiments, the first and second members are secured after bringing them into proximity. In various embodiments, the first and second members are secured with an adhesive. In some embodiments, the adhesive is cyanoacrylate. In further embodiments, the adhesive is released from at least one member. In yet additional embodiments, the adhesive is released from the members when the connective elements of the first and second members engage. In some embodiments, each member comprises two or more layers, at least one layer being porous. The porous layer is sealed by a material that provides pressure-induced porosity. In further embodiments, each member includes two or more layers, at least one layer being porous, wherein the adhesive is present in the porous layer.

In further embodiments, the two or more closure components are positioned in spaced relationship ranging from approximately 1 mm to 5 cm.

In some embodiments, the method further includes affixing the positioned members. In some embodiments, the members are affixed with an adhesive. In one embodiment, the adhesive is a pressure sensitive adhesive, and in another embodiment, the adhesive is glue.

Additional embodiments further include bringing the edges of the tissue opening into proximity prior to positioning the closure members. In some embodiments, the edges of the tissue opening are brought into proximity using a temporary closure device comprising a deformable perimeter defining an opening, the deformable perimeter having a first surface and at least one adhesive on the first surface. In additional such embodiments, the temporary closure device surrounds the tissue opening. In further such embodiments, the method further includes pressing opposing portions of the perimeter towards each other thereby exerting a force on tissue underneath the temporary closure device to bring edges of a tissue opening closer together. In an alternate embodiment, one or more closure members is placed in proximity to the tissue opening prior to the use of a temporary closure device.

Another aspect of the invention provides for a closure device for closing a tissue opening, including one or more closure components, each closure component having a member; the member having a first surface that adheres to a tissue surface proximate to the tissue opening, the first surface having at least one adhesive; and the member including an eversion element, the eversion element contoured to evert an edge of the tissue opening upon the application of the closure component. In an embodiment, the eversion element is a resilient member, for example, a spring mechanism.

In some embodiments, the adhesive is a semi-permanent skin-bonding agent. In further embodiments, the semi-permanent skin-bonding agent is a pressure sensitive adhesive. In additional embodiments, the semi-permanent skin-bonding agent is a skin glue.

In various embodiments, the member has a first layer and a second layer. In some embodiments, the closure device has a reservoir between the first layer and the second layer, the reservoir containing a second adhesive. In some embodiments, the second adhesive is cyanoacrylate. In additional embodiments, at least one layer is porous. In further embodiments, at least a portion of the porous layer is sealed by a material that provides pressure-induced porosity. In further embodiments, a second adhesive is present in the porous layer

In some embodiments, the invention provides for the use of the devices, methods, kits, and systems described herein for the treatment and/or closure of a tissue opening or in the manufacture of a device, kit or system for the treatment or closure of a tissue opening.

There are numerous advantages to the various embodiments of the invention. One advantage of some embodiments of the invention is that the wound closure components of the invention can be quickly and easily affixed to wound skin edges and aligned for proper orientation. The components are placed at intervals along a wound before closure. Paired opposing components attach to each other to bring skin edges into proximity. When opposing members are attached to each other, they form a bridge to approximate that section of the wound and provide an optimal environment for wound healing. The members provide vertical force orthogonal to the wound plane, thereby creating wound edge eversion to promote healing. In some cases, the components contain a locking mechanism to allow opposing components to form a tight bond.

Another advantage of various embodiments of the invention is that the devices and methods provide gross alignment of skin edges, fine alignment of skin edges, targeted placement of adhesive closure components, and long term bonding of the adhesive closure components.

The methods of some of the embodiments of the present invention are also advantageous because they are directed to creating a comfortable working environment and improved step-appropriate sterility during a wound closure procedure. For example, a multilayer kit may offer an instruction layer, a cleaning layer, a sterile field layer, and/or a closure layer. Each layer may be distinct to allow appropriate levels of cleanliness or sterility, availability to pour and use necessary fluids, and a clean workspace for that step of the procedure. The closure layer contains a wound closure device or system. Another advantage is that a system is also provided for post-procedural care of the healing wound.

The foregoing and other features and advantages of the invention will be apparent from the following more particular description of the embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

FIG. 1 is a schematic cross-section of a closure component.

FIG. 2 is a schematic cross-section of a closure component in which the members are joined.

FIG. 3 is a schematic top view of a closure component.

FIG. 4 is a schematic top view of a closure component in which the members are joined.

FIG. 5 is a schematic top view of multiple closure components placed at set intervals.

FIG. 6 is a schematic top view of multiple closure components placed at set intervals and in which the members are joined, bringing skin edges into apposition.

FIG. 7 is an oblique view of a closure component that provides mechanical closure and skin edge eversion.

FIG. 8 is an oblique view of a closure component that demonstrates how closure can produce mechanical skin edge eversion.

FIG. 9A is an oblique view of a closure component that provides mechanical closure, skin edge eversion, and latch release.

FIG. 9B is an exploded view of FIG. 9A.

FIG. 10 is a top view of a closure component that provides mechanical closure, skin edge eversion, and latch release.

FIG. 11 is a lateral view of closure component members that provide mechanical closure, skin edge eversion, and latch release.

FIG. 12 is an oblique view of a wound closure component that provides mechanical closure, skin edge eversion, latch release, and allows for members to be the same on opposing sides of the wound.

FIG. 13 is a view of a temporary closure device to achieve gross wound edge apposition, with a gaping laceration in its center.

FIG. 14 is a view of a temporary closure device to achieve gross wound edge apposition, deployed so that the central laceration is grossly apposed.

FIGS. 15A-15C are a representation of a device for applying closure components, assuming previous gross wound edge apposition, and an adhesive dispenser and a kit for use therewith.

FIGS. 16A-16B are a representation of a device for applying closure components which does not require previous gross wound edge apposition and materials for use therewith.

FIGS. 17A-17M are representations of a method of using a kit for a closure system including the kit, various components contained within the kit, and a method of its use.

FIGS. 18A-18D are representations of a closure device containing multiple wound closure components and a method of its use.

FIGS. 19A-19I are representations of a method of using a kit for a closure system including the kit, various components contained within the kit, and a method of its use.

FIGS. 20A-20F are representations of a closure device containing multiple wound closure components and a method of its use.

FIG. 21 is a schematic view of a male member.

FIG. 22 is an exploded view of FIG. 21.

FIG. 23 is an exploded view of a female member.

FIG. 24 is a schematic view of a female member.

FIG. 25 is a schematic view of a female member showing a latching mechanism.

FIG. 26 is a schematic view of a male member and a female member engaged.

### DETAILED DESCRIPTION

Various embodiments of the invention relate to systems and methods for closing a tissue opening non-invasively. Additional embodiments of the invention relate to devices or kits structured to improve the process of wound closure. Further embodiments of the invention also relate to systems and devices for post-procedural wound care.

As used herein, a “wound” refers to any tissue opening, whether accidental or intentional, including but not limited to, a surgical incision, accidental laceration, or other form of injury. In various embodiments the wound is a tissue opening in the skin or other tissue. The wound may be in any animal, including human and non-human animals. In some embodiments, the wound is in a mammal or a non-human mammal.

By way of a non-limiting example, in some embodiments, a closure kit contains a treatment tray. The tray can have a multi-stage modular system for controlling the treatment environment. Unfolding packages within the tray creates a procedural field while maintaining critical aspects of the closure system close at hand for rapid use. This prevents the common scenario of a cluttered table with difficult to reach instruments.

In some embodiments, the needle-less or non-invasive closure system includes a set of clip-like devices designed to rapidly and easily close a wound by mechanical means. It can be applied to even large wounds in minutes, whereas sutures, the traditional alternative, often take up to an hour for placement. Some embodiments achieve wound closure through a “skin buckle” mechanism with rapid and simple placement through combination adhesives. Each closure member is positioned on either side of opposing laceration edges and initially secured in place by a pressure sensitive adhesive similar to tape. When the device is engaged, or buckled, the wound edges are mechanically drawn together and the wound is sealed. When fastened, the device releases a semi-permanent skin-bonding agent, creating a closure that will resist migration and dislodgement. When the wound is healed, the device falls off due to normal exfoliative processes; alternatively, the device can be removed easily and painlessly at the physician's discretion using acetone or another solvent commonly available.

Functional benefits of such a wound closure system that relies on a combination skin adhesive and mechanical device to bring skin edges together include, without limitation, ease of placement and elimination of needles. For large wounds, administration of local anesthesia, such as Novocain, commonly includes several needle punctures. Each administration, assuring wound numbness, is accompanied by a burning sensation at the injection site. Because no needles are necessarily used to place this device, no local anesthesia is necessary. The elimination of needles reduces pain and emotional distress for patients and associated disease transmission risk for healthcare providers. Furthermore, because various embodiments of the closure utilize a mechanical device rather than current tape-based (steri-strips) or uni-dimensional (stitches) processes, more control of outcome is obtainable. Scientific principles show that cosmetic outcome in wound healing is largely determined by apposition and eversion created during wound closure.

Apposition is the proper alignment of skin edges to prevent gaps or mismatch. By allowing the physician to place and replace the device using a pressure sensitive adhesive before a skin bonding agent is released, better or optimal apposition can be achieved with various embodiments of the invention than many common conventional methods and devices.

Eversion is the raising of skin edges during the initial healing process. This is critically important as expected scar formation includes myofibril contraction, which ultimately draws the scar slightly below the surrounding skin level. This is often seen in surgical scars, which have divets or slight depressions in the center. Eversion prevents or at least minimizes this phenomenon by starting with slightly raised skin which lays flat after the universal process of contraction, which occurs at about 2-3 weeks after injury. The medical community generally recommends skin edge eversion to promote the least noticeable scar. Unfortunately, skin edge eversion is difficult or impossible to achieve with simple stitches or glues. More complex stitches that promote adequate eversion are time consuming to place and are used in less than one case in one hundred.

Currently, after sutures, staples, or other closure techniques, patients are often told to remove dressings within 48 hours, even though it is known that follow-on dressings can reduce scar formation. However, in an embodiment of the invention, the wound healing process is broken into three stages during which three different dressings can be applied. The first stage primarily requires a moist healing environment. A moisture-tight sealant is designed to reduce unwanted shock and promote a moist environment. The second stage, beginning 3-4 days following wound closure, often benefits from rich application of nutrients and oils. Vitamin E and other compounds can be contained within a soft dressing to promote healing and a refreshing feeling to the skin. The third stage begins 7-8 days following wound closure. Although the skin has largely healed at this point, there are still compounds that can reduce scarring. A combination of beneficial substances can be included within the third stage dressing to maximize scar softness and reduce discoloration.

After thorough cleaning of the wound edges, the wound closure components are affixed to the skin. For example, attachment processes for the closure components include adhesive backing, pre-placed skin glue, skin staple, or other adherent mechanism. Once applied to a skin edge, several techniques may be used to allow fine-tuning of placement of the closure components or their members, including but not limited to, sliding tracks on the attachment piece, a swivel element, such as for example a swivel element within the piece, or a replaceable adhesive back.

In various embodiments, the members of a wound closure component are placed opposite each other on either side of a surgical wound. They may be approximated (drawn together using connective elements), such as, for example, an interlocking system or other fastening device. When the components are brought into approximation, the attached portion of the wound should similarly close. In some embodiments, the closure should provide for as little space as possible between closure components and skin. In some embodiments, the release of the attached members should be possible, such as, for example, either through a protected release mechanism or through a release tool.

In some embodiments, components are placed in spaced relation, such as for example at intervals, along the long axis of the wound. These intervals can be long to allow drainage in wounds with high risk of infection or short for wounds which require a more controlled approximation. The intervals may be regular or irregular in length. All components may be placed and then the members connected (such as for example when the connective elements are engaged) to close the wound or the wound may be closed incrementally as each component pair of members is added.

In some embodiments, the coupled closure device (including both members when engaged) is about 5 mm in length and 4 mm in height (small size). In other embodiments, the coupled closure device is about 10 mm in length by about 5 mm in height (medium size). In further embodiments, the coupled closure device is about 30 mm in length by about 10 mm in height (large size). In various embodiments, the aspect ratio (length versus height) may range from approximately 1:1 to approximately 3:1. In some embodiments, the aspect ratio of a small closure device is about 1:1, and in additional embodiments, the aspect ratio of a medium and/or large closure device is about 3:1. In one embodiment, the aspect ratio is about 2:1.

Non-limiting examples of materials contemplated for the wound closure components include plastic, metal, and other polymers, or combinations thereof. In some embodiments the wound closure components are bioabsorbable. In one embodiment, the components will be light, strong, and waterproof. It is possible that with some materials, the components may be cleaned and sterilized for reuse. The components may be applied under sterile or medically clean conditions depending on the type of wound and according to the best judgment of the healthcare provider. In some embodiments, the components (or one or more members thereof) contain an agent, such as for example a chemical, to detect and signal impending infection. Alternatively, in some embodiments, such an agent, may be added to or above the closure during or after the procedure.

Such components can be manufactured using methods including but not limited to plastic injection molding or other technology known in the art. The use of identical members simplifies manufacturing.

In some embodiments, the components provide a vertical force orthogonal to the wound plane, thereby creating wound edge eversion to promote cosmetic healing. This may be accomplished through an upward curve of the skin-apposing surface of each member of each component, a system that mechanically raises the component undersurfaces when paired components are joined, or another mechanism. In some embodiments, the wound closure components are multiple, paired, independent, external, low-profile and provide skin edge eversion and tight paired locking.

Advantages of this device and method include, but are not limited to, the needleless technique, the ease of the technical procedure, the excellent skin edge apposition, the beneficial wound healing environment that is created, the efficacy possible for large wounds, and a high degree of patient comfort during and after the procedure.

In one embodiment, the wound closure component members may be easily placed and repositioned on the skin via a pressure sensitive adhesive bonding. These members may be set in appropriate position before releasing a second adhesive bonding agent that creates a long-term bond between the member and skin. For example, without limitation, a long-term tissue-bonding agent is cyanoacrylate. Other biological glues can also be used, such as, for example, thrombin gel.

In various embodiments the opposing members may interlock mechanically with a male-female latch. A single member may have both latches so that the male faces the female aspect when the members are set facing each other. In this way, the same member type may be used on both sides of the wound (they may be identical) and they will interlock with each other. The members may have a tapered edge, including but not limited to a beveled contact edge, so that the skin near the wound interface is raised mechanically upon attaching the closure components. In various embodiments, a long-term skin bonding agent may be released automatically upon connecting the opposing closure members; it may be released manually by the health care provider from a glue container (such as for example a dispenser) within the component, or it may be manually added and allowed to diffuse along the underside skin interface (between the first surface and the skin) of the closure member.

Various devices may be used to support easy and accurate placement of the wound closure components. One such device provides gross wound edge apposition. In one embodiment, this device is a temporary closure device, such as for example a deformable perimeter defining an opening. In some embodiments, this device has a pressure sensitive adhesive backing which can be manipulated and will hold its shape. In this way, once it is attached to skin around the wound, manipulation of the object will bring and hold skin edges (such as for example wound edges) in gross apposition. In one embodiment, the device is a plastic oval with an internal thick wire and an underside pressure sensitive adhesive. In another embodiment, the device is pressure sensitive adhesive handles which are placed on either side of the wound and allow a healthcare provider to use one hand to squeeze edges of the wound together. By way of non-limiting example, in some embodiments, two separate handles having pressure sensitive adhesive on at least one surface are placed with one handle on either side of the tissue opening before closing the opening. A healthcare provider can pull and/or push these handles together using a single hand, such as, for example, using the thumb and forefinger of one hand. Thus, gross apposition of the edges of the tissue opening can be achieved with one hand. Therefore, as one hand brings the edges of the tissue opening together, the other hand can be used to place a closure device, such as, for example, an adherence-based closure device, allowing a single healthcare provider to close a tissue opening with two hands. In another embodiment, a device supports placement of wound closure components once gross wound edge apposition has been attained. In some embodiments, this device allows closure components to be placed and releases a long-term skin bonding agent, such as for example cyanoacrylate, to create a semi-permanent bond.

In various embodiments, a single device may be used to provide gross wound apposition and to apply wound closure components. In some such embodiments, one element of the device brings skin edges into gross apposition. By way of a non-limiting example, this element may be pressure sensitive adhesive pads or other tacky or sticky pads which provide friction on the skin surface. In some embodiments, these pads may be mechanically controlled by the device to pull toward each other, bringing skin edges into gross apposition. In some embodiments, another element of the device allows fine-tuning and placement of wound closure components. Fine-tuning may be accomplished through mechanisms, including but not limited to, a mechanical roller switch or another mechanism. In various embodiments, placement of closure components may be accomplished by lowering a closure component having pressure sensitive adhesive backing onto skin over a grossly apposed wound. In one embodiment, another component of the device releases a long-term skin-bonding agent to create a semi-permanent bond between closure component and skin.

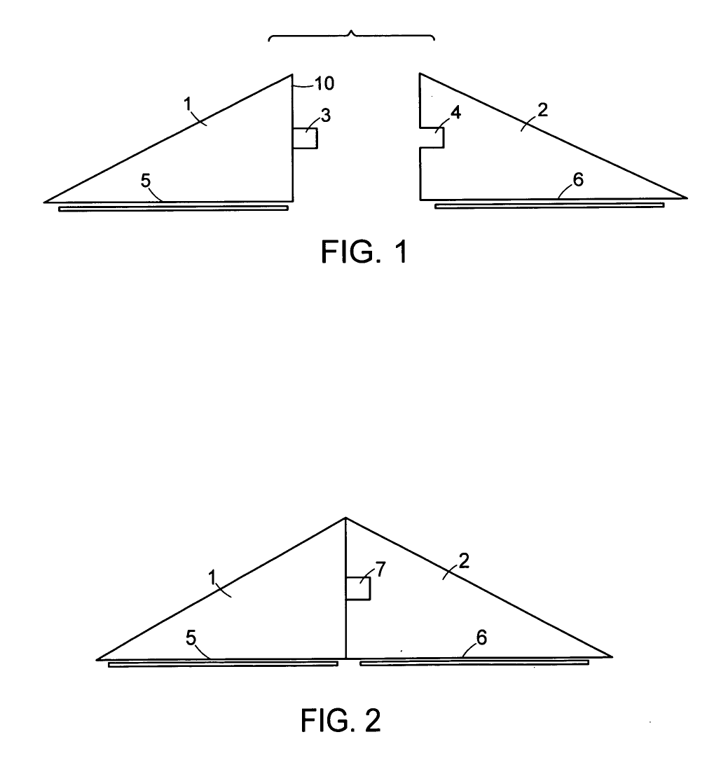

FIG. 1 depicts one embodiment of the invention, which includes two locking wedges. First member 1 contains an adhesive undersurface 5, in which an adhesive is attached to or on a first surface 5 of the member, to attach to the skin edge, and a male locking component 3, which is a connective element on a second surface 10 of the member substantially orthogonal to the first surface. The paired opposing member 2 contains female locking component 4 and adhesive undersurface 6.

The connective elements may differ in various embodiments. In some embodiments, the connective elements are interlocking and may comprise a locking mechanism. By way of a non-limiting example, the connective elements may include, but are not limited to, a ball in socket structure, hook and loop attachment, clasp, magnet, lace, ratchet, suture ligature, snap system, photobonded, non-photobonded, staple, resilient member (such as, for example, a spring), or other locking mechanism. In some embodiments, the connective elements are adhesives, including but not limited to, glue or tape. In one embodiment, the connective elements are a ball in socket structure due to the strong grasp and ease of closure of this type of mechanism. In some embodiments, the first member has a locking member extending outward from the second surface of the first mechanism, and the second component has a receiving member.

Adhesive undersurface 5, 6 may be a peel-to-expose adherent backing, a pressure sensitive adhesive, a rough surface that attaches to a glue-prepared skin edge, or another adherence mechanism. In some embodiments, at least one layer has positioned therein channels and/or perforations, or uses the inherent porosity of the materials, such that the layer is in communication with the first surface.

In some embodiments, the members comprise one or more layers, and at least one layer is a porous material. In additional such embodiments, the porous material holds the second adhesive, such as for example, cyanoacrylate. In addition, reservoirs for the second adhesive could also be used in conjunction with the porous material. In some embodiments, the second adhesive is dispensed intentionally as the connective elements become engaged or after the connective elements are engaged in the latching or locking step. In some embodiments, the second layer is sealed by a material that provides pressure-induced porosity. In additional embodiments, this layer is a selectively-permeable layer, which can control or regulate, among other things, the direction of flow of a material. In some embodiments, such a material is similar to a vapor barrier used in outdoor garments that allows moisture to travel in one direction.

FIG. 2 shows the closure component of FIG. 1 in which the second surface of the first member has been brought in operative relation to the second surface of the second member such that the connective elements engage each other. In this embodiment, the joined members form a tight bond 7 and are shown forming a pyramidal low-profile shape.

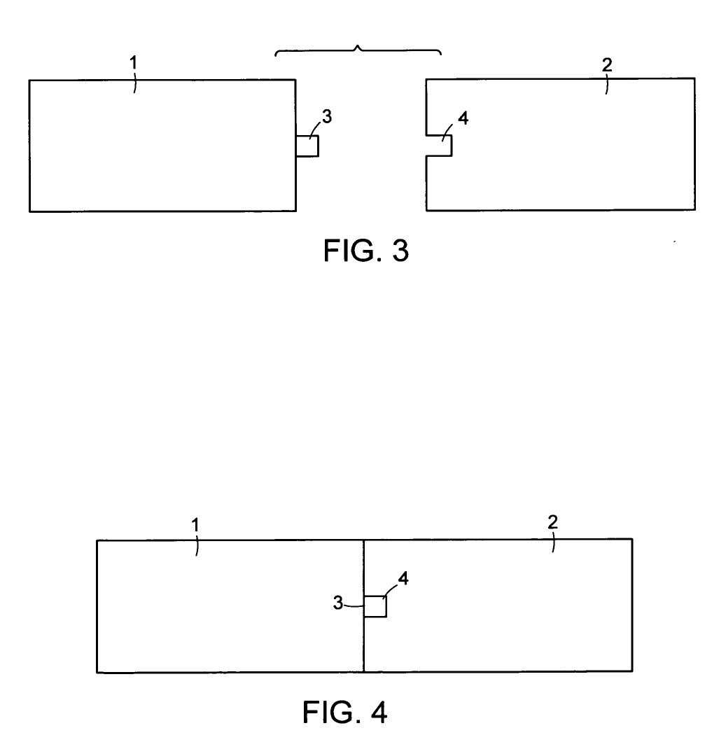

FIG. 3 depicts a top view of the closure components of the embodiment of FIG. 1. FIG. 4 shows a top view of the embodiment of FIG. 2 in which the closure members are joined by the connective elements.

FIG. 5 shows a series of closure components. First member 1 is shown approximately opposite a second member 2, forming a pair. The pairs of closure components are shown placed at fixed intervals, which are of approximately equal length, along free skin edges 8, 9 of a wound. For small, complex tissue openings, the pairs of closure components can be positioned from about 0 to about 3 mm apart. For medium size tissue openings, the pairs of closure components can be positioned from about 0 to about 1 cm apart, and for large and/or complex tissue openings, the pairs of closure components can be positioned about 0 to about 5 cm apart along the edge of the tissue opening. In one embodiment, the interval length is about 3 mm.

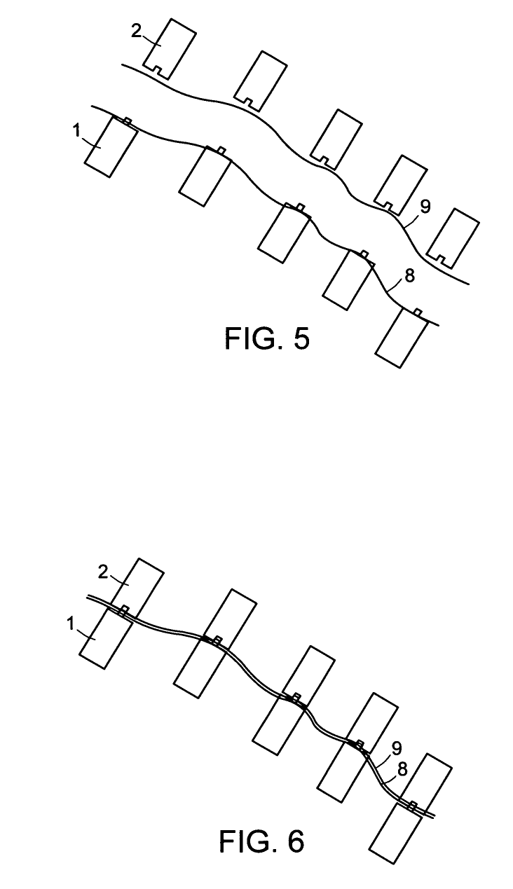

FIG. 6 depicts the series of closure components of FIG. 5 in which the paired members are joined to bring together skin edges into close apposition, promoting improved healing and reduced scarring. Each of the pairs of members has been brought in proximity to each other such that the connective elements are in operative relation and become engaged. This causes the underlying skin to be pressed together such that first wound edge 8 is brought in proximity to second wound edge 9. The paired members can either be joined as each pair is placed or after all components are placed along both edges of a wound at the discretion of a healthcare provider. It is understood herein that the mechanism for bringing the connective elements in operative relation and/or engaging them could be done manually by a healthcare provider, using a tool, such as, for example, a handheld tool using any other appropriate tool or instrument or using a combination thereof.

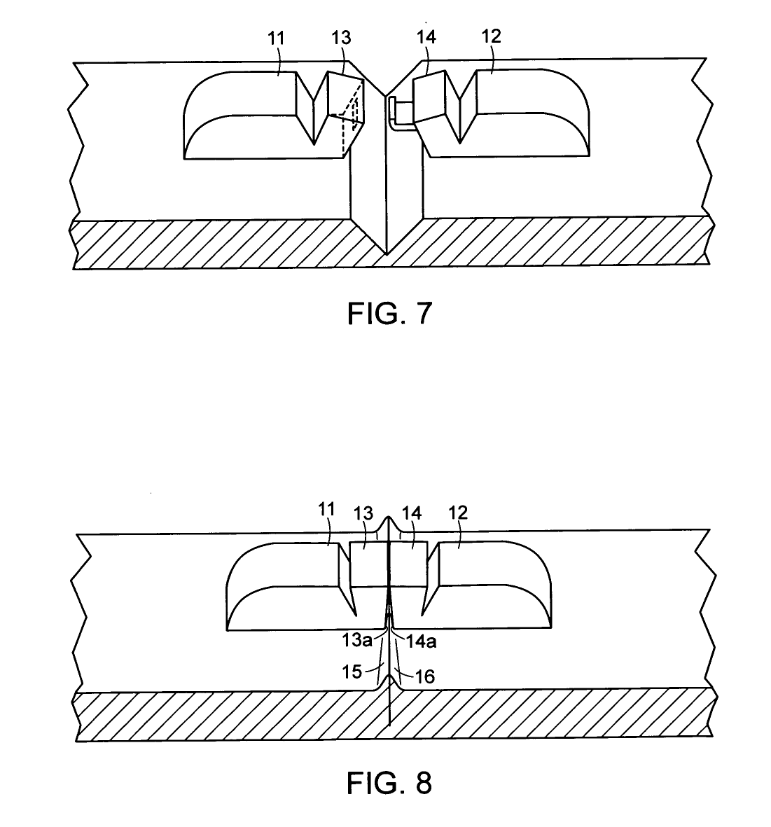

FIG. 7 depicts one embodiment of the invention, which allows for mechanical locking of wound closure component members as well as mechanical skin edge eversion. Shown here, two beveled edges, upon apposition, will cause upward traction and create local edge eversion. In the depicted embodiment, cutaway sections 13, 14 of closure members 11, 12 facilitate tissue opening edge eversion. In this embodiment, cutaway sections 13, 14 provide a jointed or flexible device that allows the turning or pivoting of a part on a stationary frame, within each member. In some embodiments, the transition region is a hinge. In additional embodiments, the hinge is cut on a diagonal such that the angle between the plane defining the bottom surface and the surface having the connective elements is about 45°. When the connective elements become engaged, any movement, for example, upwardly, occurs within either or both members, resulting in the mechanical eversion of the tissue opening edges. Thus, the transitional region defined as a hinge is positioned such that beveled edges 13a, 14a of the closure device apply pressure by pressing against tissue adjacent to and/or at the edge of the tissue opening and force the tissue nearest the tissue opening edge upward when the connective elements become engaged to lock first member 11 to second member 12. In some embodiments, when the tissue closure device is used on the skin, both the epidermal and dermal layers are forced upwards to evert the tissue opening edges. As shown in FIG. 8, first member 11 and second member 12 are brought together such that the connective elements are in operative relation to each other. This forces beveled edges 13a, 14a to draw upward as the connective elements engage. This action simultaneously draws attached skin 15, 16 upward, resulting in eversion of the wound edge. As depicted in FIG. 8, cutaway sections 13, 14 have moved relative to their position in FIG. 7.

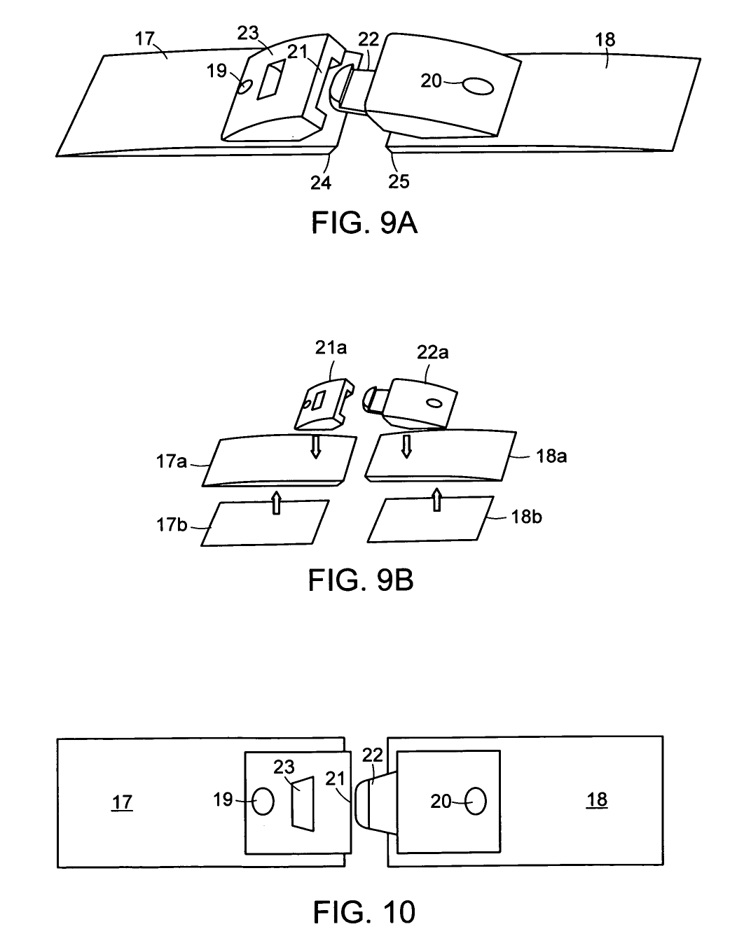

FIG. 9A shows one embodiment of the invention, which allows for simple mechanical closure, mechanical skin edge eversion, and closure release through a push button release hole. The two members have bases 17, 18, which attach to skin at the undersurface (or first surface). The latch mechanism represented here shows female end (connective element) 21 juxtaposed near or in proximity to male end (connective element) 22. This embodiment also includes latch release (closure release mechanism) 23 to unlock the members. In some embodiments, the members may be unlocked and relocked by engaging the connective elements again. Also shown are holes 19, 20, which can be used for application of an adhesive. In some embodiments, a long-term skin-bonding agent is dropped or permitted to be released through these holes. In this embodiment, beveled edges 24, 25 are used to force mechanical skin edge eversion. FIG. 9B depicts an exploded view of an embodiment such as that shown in FIG. 9A. As illustrated in FIG. 9B, a component, in some embodiments, has layers: two members 21a, 22a (such as, for example, a plastic clip), elastomeric base 17a, 18a, and adhesive 17b, 18b such as, for example, a pressure sensitive adhesive coating.

FIG. 9B is an exploded via illustrating the components described with respect to FIG. 9A.

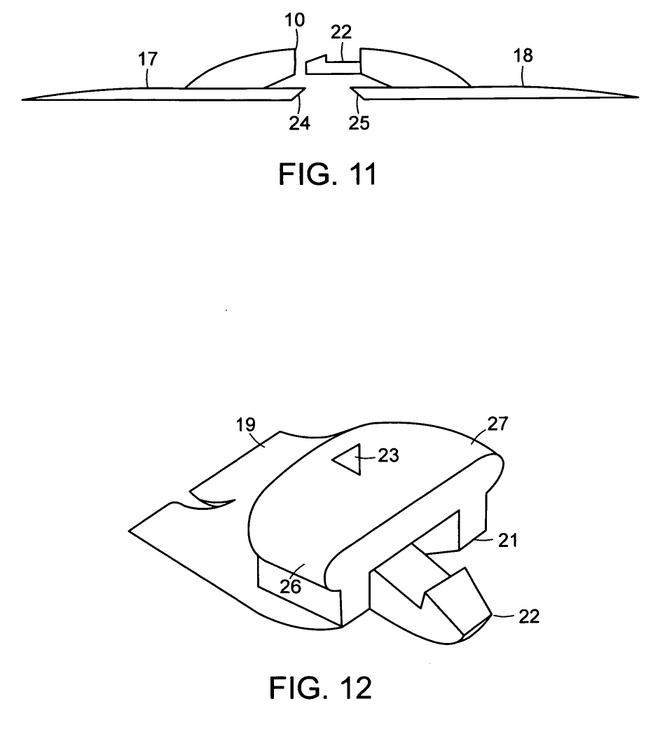

FIG. 10 shows a top view of the embodiment of FIG. 9A, and FIG. 11 shows a side view of the embodiment of FIG. 9A. Second surface 10 is not a flat surface in this embodiment. Beveled edges 24, 25 at the transitional region between first surface and second surface of the member is more clearly depicted in FIG. 11.

FIG. 12 shows an alternate embodiment of the invention, which allows for simple mechanical closure, mechanical skin edge eversion, closure release, and design symmetry. In this embodiment, female latch mechanism 21 and male latch mechanism 22 are side by side on the same member. If a similar piece were placed on the opposite side of a wound, its conformation would be the mirror image of the opposing member, allowing for each male latch to interface with (or engage) the female latch on the opposing member. This eliminates the need for differently structured members on each side of the wound, allowing for greater ease of manufacture because all of the members are identical to the extent possible. The embodiment depicted here also contains wings 26, 27, or protrusions extending from the member. In some embodiments these protrusions are substantially parallel to the first surface. Such protrusions allow for easier handling. The embodiment depicted in FIG. 12 also includes hole 19 (an opening provided in plane that is substantially parallel to the first surface and spaced apart from the first surface extending through to the first surface) for introduction of an adhesive, such as for example, a second adhesive or semi-permanent bonding agent. This figure also depicts a latch release mechanism (closure release mechanism) 23, which can be used to disengage the first and second components. Disengaging the first and second members can be used, by way of a non-limiting example, to facilitate removal of the members after the wound has healed or to facilitate repositioning one or more of the members.

In some embodiments, the invention provides a device for closing and maintaining closed a cutaneous wound in order to promote healing, said device having a plurality of pairs of small independent members which can be placed on and bonded to opposing skin edges of a wound at set intervals; the paired members contain a mechanical locking process, allowing opposing members to be joined to bring skin edges into apposition within a specific section of the wound.

In additional embodiments, the device further includes an agent bonding the closure component to the skin, such as for example adhesive or glue. In further embodiments, the locking mechanism that attaches members at opposing edges of a wound is mechanical rather than adhesive. In some embodiments, the locking mechanism includes male and female pieces which join opposing members. In one embodiment, the locking mechanism includes a ball in socket structure.

In further embodiments, a sliding lock runs in a direction longitudinal to the wound axis and holds opposing wound members together. In additional embodiments, a release button or tool exists to release locked members. In some embodiments, the locking mechanism is reflexive, with the lock being symmetric along the long axis of the closure member, allowing for a single design to function on either side of the wound. In various embodiments, the components are narrow and low profile to limit obtrusiveness and risk of accidental dislodgement. In additional embodiments, the members have a fine-tuning placement mechanism allowing their position to be slightly altered once affixed to the skin, and in some such embodiments, the fine-tuning mechanism consists of a pressure sensitive adhesive undersurface which can be replaced until proper positioning is attained. In further embodiments, the fine-tuning mechanism consists of a small rail, ball bearing, or hinge allowing a degree of flexibility to the closure member before or after attachment to the opposing member. In additional embodiments, the flexible fine-tuning mechanism has an independent locking system so that once appropriately placed, the member can be made immobile.

In some embodiments, the independent locking system is mechanical. In additional embodiments, the independent locking system is a strong adhesive agent that prevents accidental removal.

In additional embodiments, a mechanism elevates the outer lip of the skin edge, producing wound edge eversion to improve cosmetic outcome. In some embodiments, the wound edges are elevated by adhering to an upwardly curving lip on the skin-apposing surface of the closure member. In further embodiments, the wound skin edges are elevated by member undersurfaces that raise mechanically when opposing members are locked together. In some embodiments, two beveled member edges brought into apposition mechanically force the undersurface of the closure member upward.

In further embodiments, a chemically impregnated material can be placed over a wound to recognize and signal an impending infection. In some embodiments, the chemical reagent which detects and signals impending infection is incorporated within a wound closure device. In additional embodiments, the chemical reagent which detects and signals impending infection is external, but made to integrate with or lay within or around the closure device.

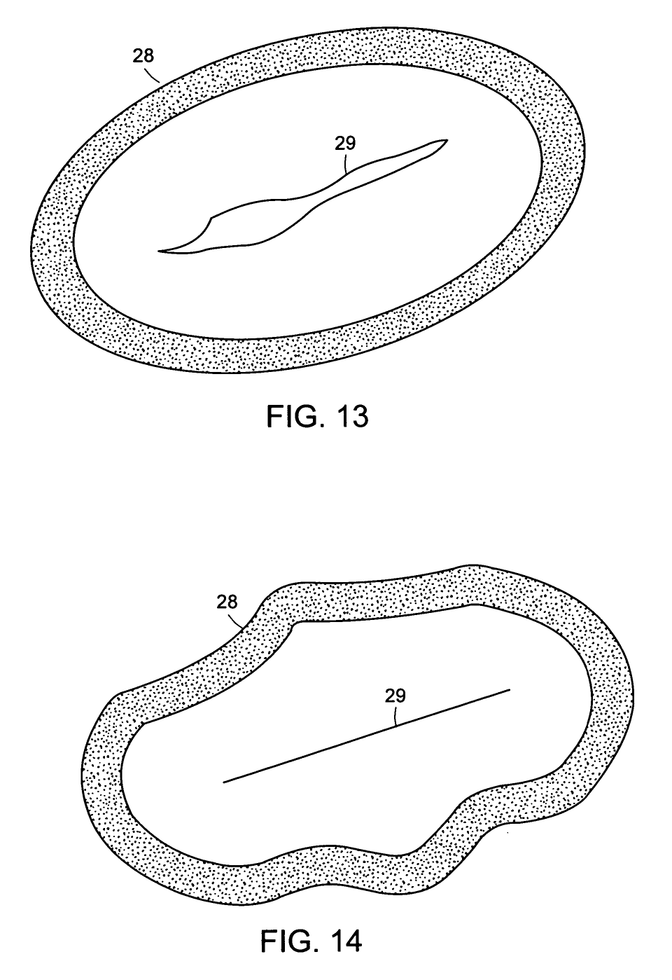

FIG. 13 shows one embodiment of a compliant, temporary closure device of the invention, which is used to achieve gross wound apposition. In this embodiment, the device has deformable perimeter 28, which is oval in shape. The deformable perimeter defines an opening that surrounds wound 29. In some embodiments, the deformable perimeter also has at least one adhesive on at least one surface in contact with the tissue surface.

FIG. 14 depicts the embodiment of FIG. 13 in which the deformable perimeter has been manipulated to close wound 29 by bringing the edges of the wound into proximity. The compliant perimeter in this embodiment maintains its shape after being manipulated in order to hold the edges of the wound closed.

In various embodiments, the invention provides device for achieving gross wound edge apposition having a control piece or control pieces which attach to the healthy skin around a wound through pressure sensitive adhesive at its undersurface and a mechanism to bring aspects of the control piece on either side of a wound together, thereby pushing the edges of the wound into apposition.

In some embodiments the control pieces are situated on either side of a wound and contain a single grip to allow easy holding or pushing. In various embodiments, the apposition mechanism is a healthcare provider gripping the pieces with opposing thumb and finger to squeeze them together.

In further embodiments, the control piece is a single oval which surrounds the wound. In additional embodiments, the oval holds its shape to maintain skin edge apposition once appropriately positioned. In some embodiments, the oval contains a thick wire within it to help it hold shape. In some embodiments, the apposition mechanism is a healthcare provider manipulating sections of the oval until skin edges are pushed toward each other into apposition.

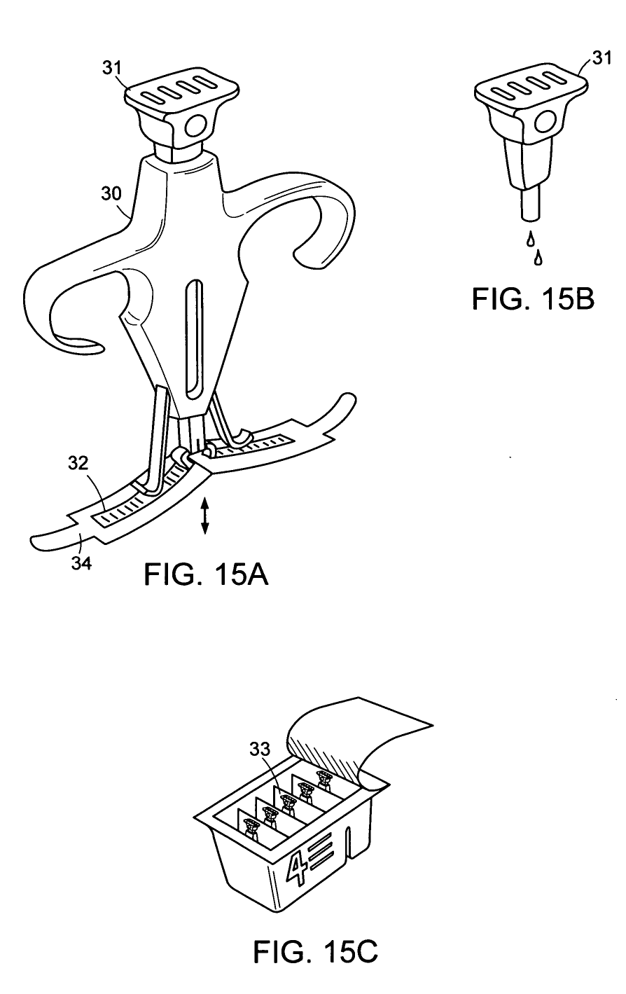

FIGS. 15A-15C illustrate an embodiment of the invention, which is a device that applies wound closure components. FIG. 15A shows one embodiment in which applicator device 30 contains a handle or handles to support easy grip. Applicator device 30 grips a version of a wound closure component 32, and contains plunger and reservoir mechanism 31 to release cyanoacrylate onto, into, or through closure component 32. In this embodiment, closure component 32 also contains peel-away backing (adhesive backing) 34. The plunger and reservoir mechanism 31 for dispensing cyanoacrylate is shown separately as a cyanoacrylate dispenser in FIG. 15B. Additionally, as shown in FIG. 15C, kit 33 may also be provided, which may contain additional closure components 33 for convenient retrieval and placement.

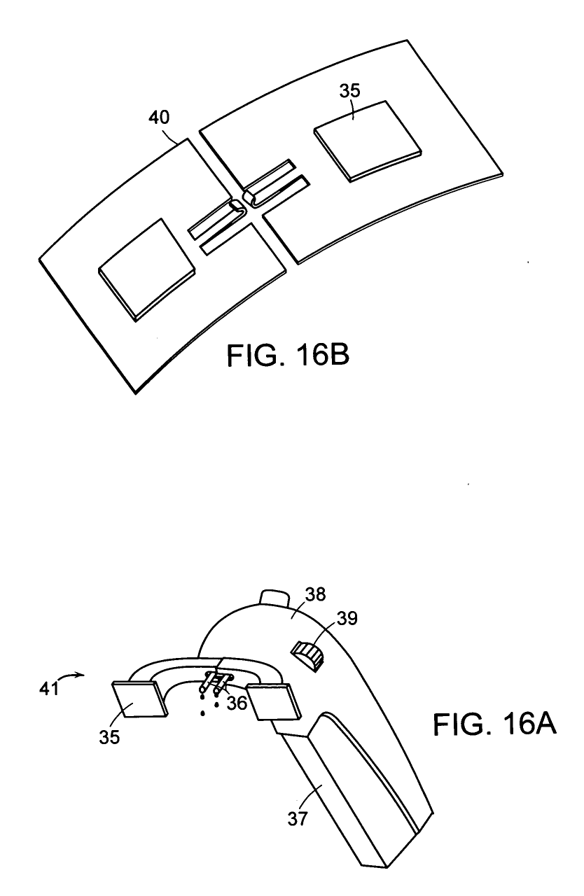

FIGS. 16A-16B illustrate one embodiment of the invention which includes placement device (or applicator device) 38, which provides gross wound apposition, wound closure component placement and fine tuning, and bonding agent release. One embodiment of applicator device 38 contains sticky pads 35 which can be placed on either side of the wound and brought together by squeezing grip 37 to achieve gross wound apposition. Once the skin edges are apposed, the placement of the wound closure component is fine tuned with a mechanical roller grip 39 until horizontal displacement is appropriate. In additional embodiments, displacement can be horizontal and/or vertical. When grip 37 is fully closed, the wound closure component is released on the skin of the wound. In some embodiments, this is accomplished using a release mechanism 36. A long term skin bonding agent may be released by the same mechanism. A kit shown in FIG. 16B containing closure components 40 may contain only the components or may also contain replaceable sticky pads 35 for the gross alignment process.

In various embodiments, the invention provides for a device for placing wound closure components having a mechanism to hold, lower, and release said closure components, and a mechanism to affix closure components in place. In some embodiments, the closure components are lowered with a hand grip. In further embodiments, the closure components are affixed with self contained pressure sensitive adhesives. In additional embodiments, an independent mechanism releases a long-term skin-bonding agent once the components are in place. In one embodiment, two sticky surfaces are placed on either side of the wound and mechanically drawn together to provide gross wound edge apposition prior to wound closure component placement. In some embodiments, the device allows fine-tuning of the closure component in the transverse and/or longitudinal direction prior to placement.

In further embodiments, the applicator device or other tool for use with the closure devices include one or more light-based guides (or mechanism for a light-based guide). In some embodiments, these guides are projected from the hand-held tools used for placing the device on the skin. In various embodiments, the light is produced in a pattern to aid the placement of the applicator device, tool, and/or closure component. By way of nonlimiting example, in some embodiments, one edge of the light pattern is cast on the edge of the tissue opening and the other edge of the light pattern is cast the appropriate distance from the wound for the edge of the device. In one embodiment, when the applicator device is positioned such that the light pattern aligns with the edge of the tissue, the applicator device is appropriately positioned for placement of the closure component. A further embodiment includes a mechanism for adjusting the light pattern based on the size of the closure component or other factors.

In some embodiments, a wound closure kit offers a systematic and appropriate layering of needed items to provide ease of use and improved step-appropriate sterility during closure. In various embodiments the kit may include at least one of an instruction layer, a cleaning layer, a sterile field layer, and a closure layer.

The instruction layer may include, by way of non-limiting example, a sturdy card with wound care instructions to be handed to the patient. This card would be sturdy, waterproof, of easy form to keep and provide reference, and provide suitable information regarding wound care, medical warning signs, and closure material removal. The card would be a timesaving technique for the provider and a mnemonic for the patient.

The cleaning layer may offer, by way of non-limiting example, a watertight cup to hold saline and a non-splash syringe to draw up saline and flush the wound. The layer may offer an option of access from outside the kit through a peel-away external cover so that fluid can be introduced into one section before the entire kit is opened and exposed.

The cleaning layer may be pulled off the kit to reveal a sterile field layer, which may include, by way of non-limiting example, sterile gloves on top and a sterile field below to allow the set up of a sterile procedure.

At the appropriate time in the procedure, the closure layer may be exposed. The closure layer contains, by way of non-limiting example, the wound closure system. This area may include one or more of the above-described components or another closure device or system.

In some embodiments, there may be no need for a sterile layer and the wound closure may be carried out under clean but not sterile conditions.

Advantages of the described kit include, but are not limited to, the ability to set up the entire kit including, without limitation, to added fluids and other substances in advance, clean and independent layers for each part of the wound closure procedure, and appropriate containment and display of instruments needed for each aspect of the procedure. Aspects of the procedure taken into account by the kit include, but are not limited to, those generally neglected by other kits, such as for example patient instruction, thorough wound irrigation, the need to lay out substances before donning sterile gloves, and an effective final wound dressing.

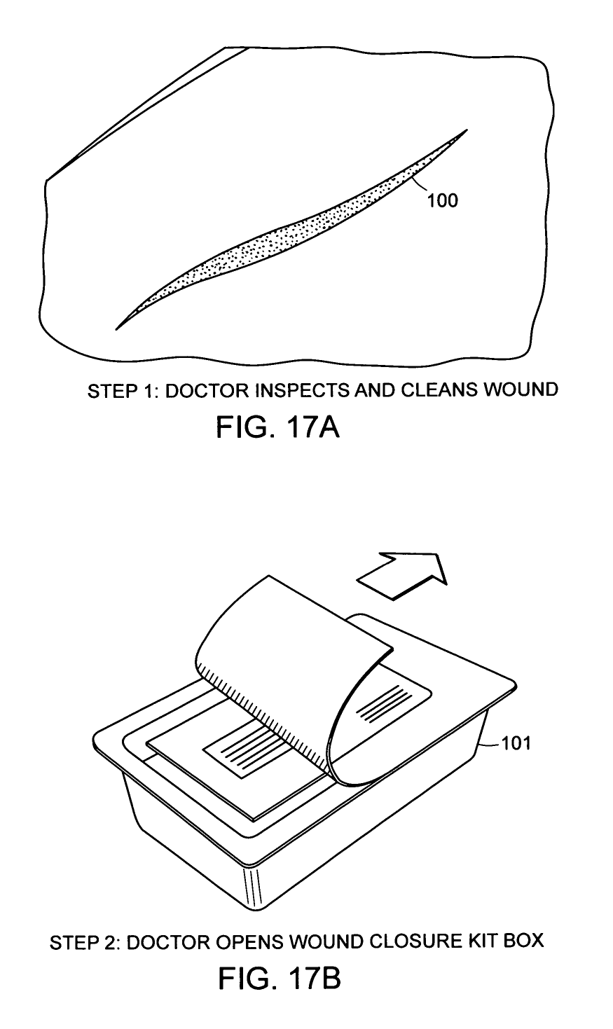

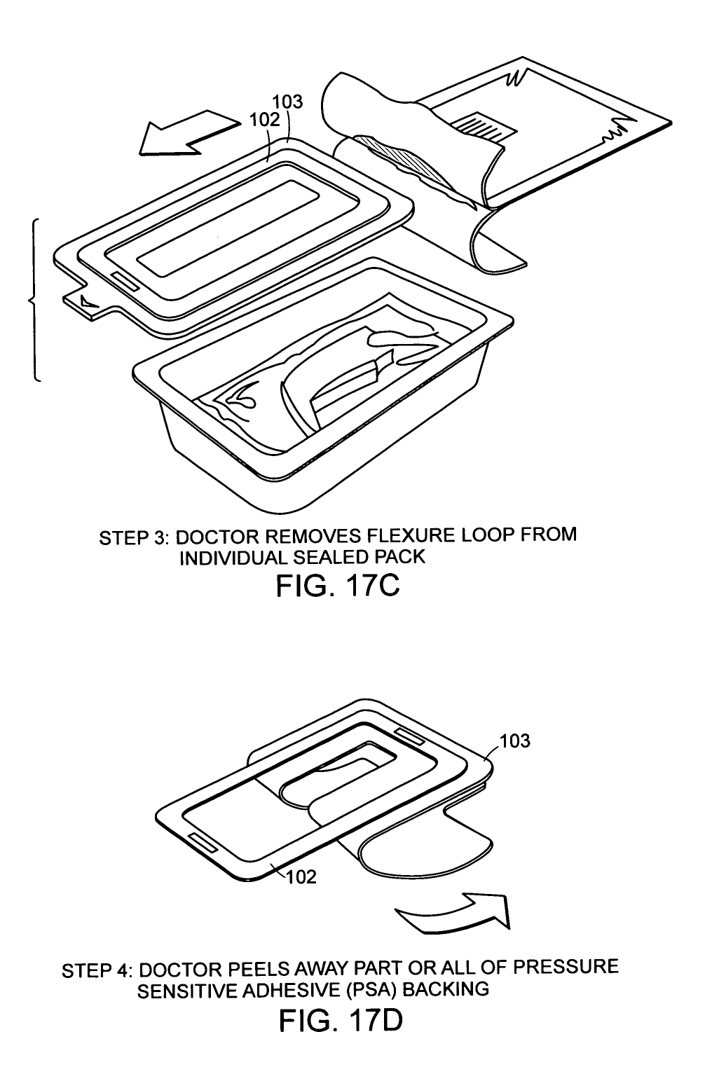

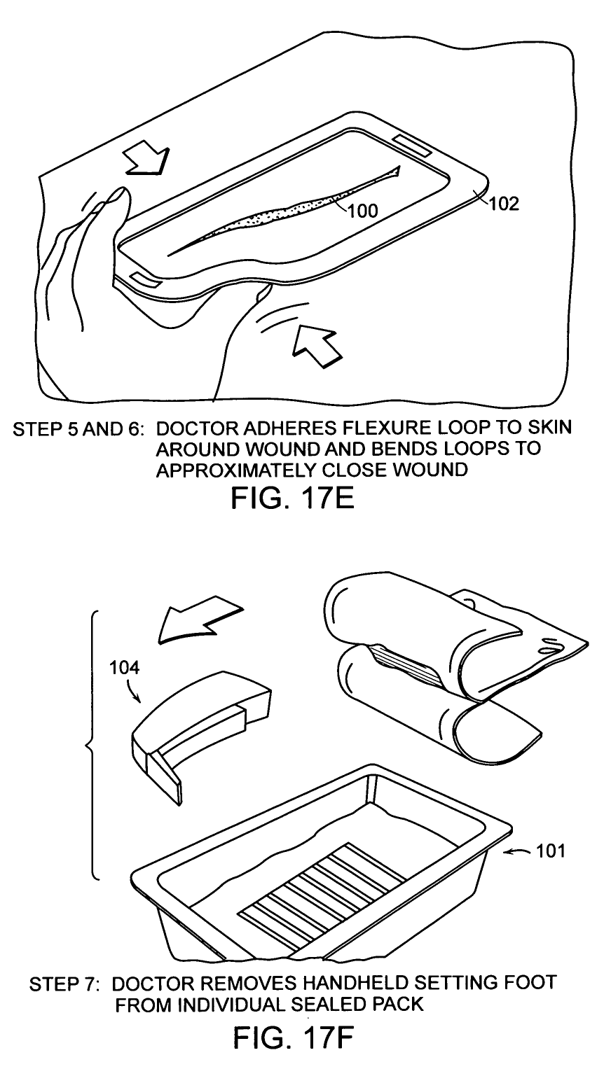

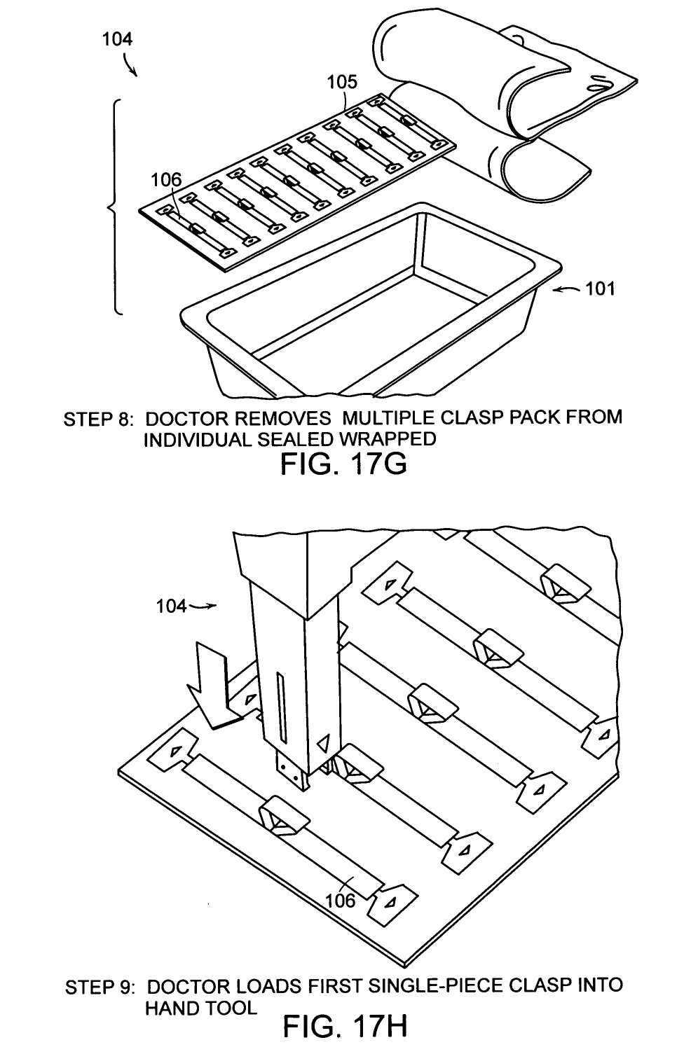

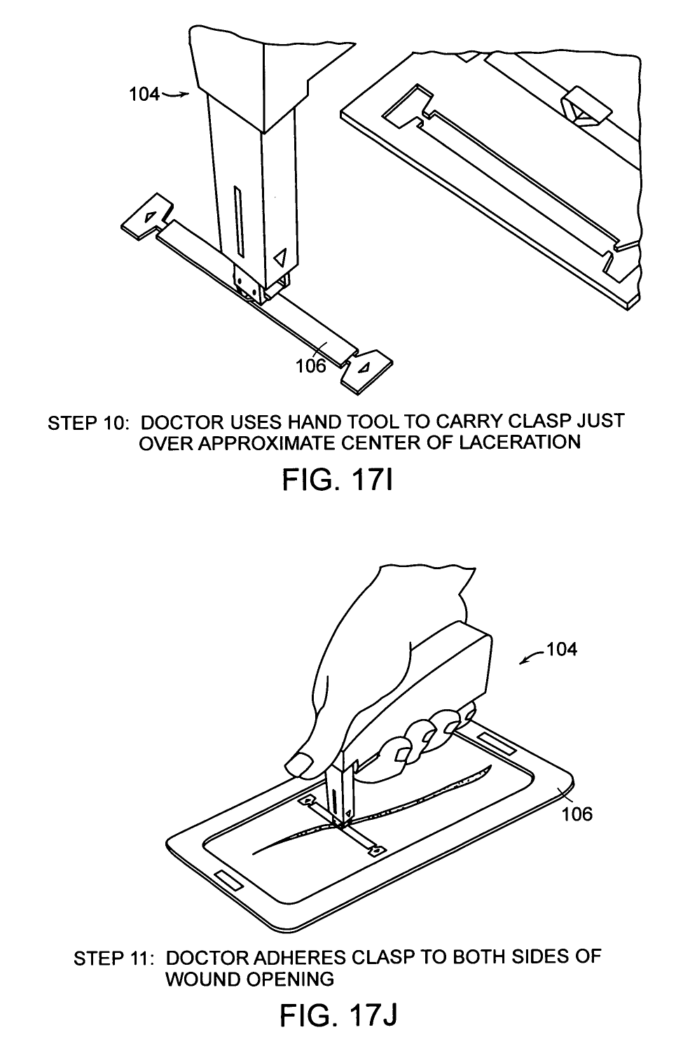

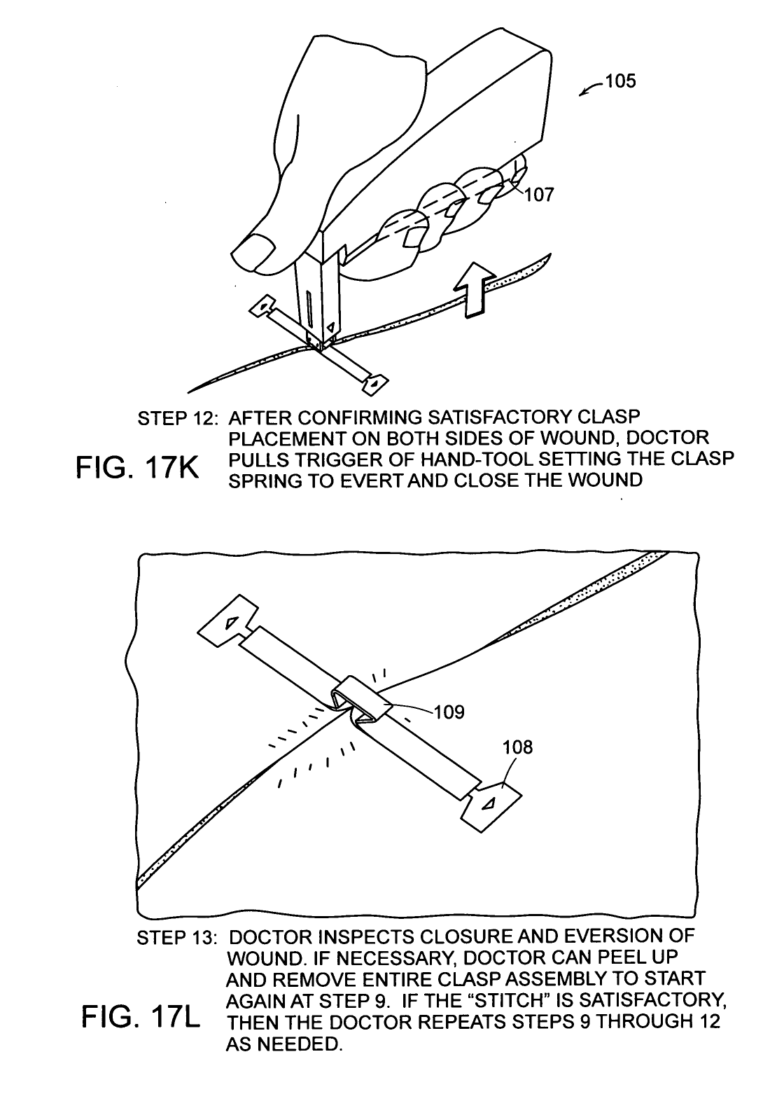

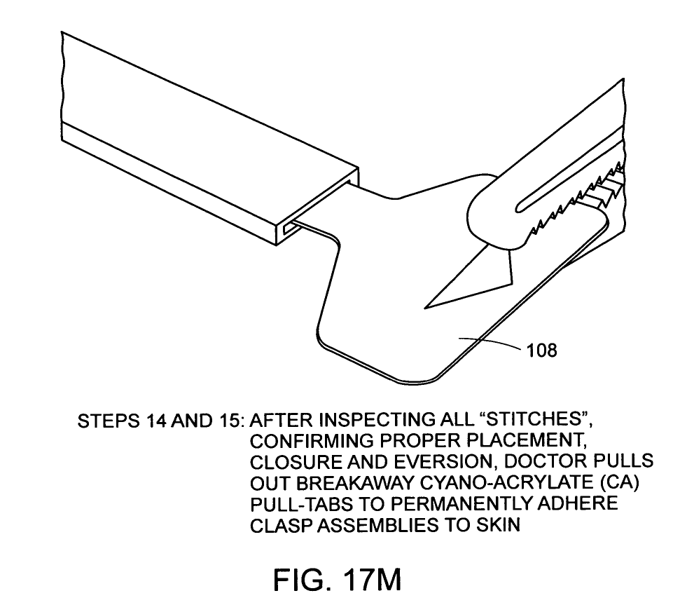

FIGS. 17A-17M illustrate a method using a kit for a wound closure system including the kit, various components contained within the kit, and a method of its use. FIG. 17A shows wound 100. The doctor or health care provider inspects and cleans the wound. In FIG. 17B the doctor opens kit 101 for closing a tissue opening by removing a cover from the kit. In this embodiment, the kit is a wound closure kit box. FIG. 17C depicts the removal of temporary closure device 102 (such as for example a flexure loop) from an individual sealed pack. Temporary closure device 102 is shown with pressure sensitive adhesive backing 103. In FIG. 17D, adhesive backing 103 is removed from temporary closure device 102 exposing the pressure sensitive adhesive. The doctor may remove part or all of the pressure sensitive adhesive backing. The doctor adheres the temporary closure device to a tissue surface around a tissue opening, which is depicted in FIG. 17E as wound 100 in a skin surface, and bends the temporary closure device to approximately close wound 100. The doctor then removes applicator device 104, which in the embodiment depicted in FIG. 17F is a handheld setting tool, from an individual sealed pack in kit 101. As shown in FIG. 17G, the doctor also removes multiple closure pack 105 (such as for example a multiple clasp pack) from an individual sealed wrapper which was in kit 101. In this embodiment, multiple closure pack 105 contains multiple closure components 106. FIG. 17H shows applicator device 104 being loaded with first closure component 106 such that a first single-piece clasp is being loaded into the hand tool. FIG. 171 shows the doctor using application device 104 (hand tool) to carry the clasp just over the approximate center of the laceration. As shown in FIG. 17J, the doctor uses applicator device 104 to adhere clasp 106 to both sides of the wound opening. After confirming satisfactory placement of the clasp to both sides of the wound opening, the doctor pulls trigger 107 of hand tool 104, which sets the clasp spring to evert the wound edge and close the wound as shown in FIG. 17K. FIG.17L shows the placed closure component with the wound closed and eversion of the wound edge. As depicted in this embodiment, the closure component includes bridging member 109 and pull mechanism 108 (depicted in this embodiment as breakaway pull tabs). The doctor inspects the closure and eversion of the wound edge. If necessary, the doctor can peel up and remove the entire clasp assembly to start again with the process shown in FIG. 17H. If the placement and closure making the “stitch” is satisfactory, the steps shown in FIGS. 17H-17L can be repeated as needed to close the wound. After inspecting all the “stitches” confirming proper placement of the closure components, closure of the wound, and eversion of the wound edge, the doctor pulls out breakaway cyanoacrylate pull-tabs 108 to semi-permanently adhere the clasp assemblies to the skin.

Various embodiments of the invention provide for a method of closing and promoting healing of a wound without use of stitches, staples, or strips, said method including the steps of positioning elevated wound closure members on opposing edges of a skin wound; fine-tuning the position of each opposing closure member to allow for optimal wound apposition; and locking the opposing members in place before or immediately after bringing together a portion of the skin wound.

In some embodiments of the method, opposing wound closure components are placed at set intervals along the longitudinal axis of a wound. In additional embodiments, the intervals can be wide with large gaps to allow wound drainage or can be narrow with small gaps to improve skin edge apposition. In further embodiments, opposing wound closure members are fixed to the skin using adhesive material or glue. In some embodiments, a short-term adhesive agent provides holding strength during initial member placement and a long-term adhesive agent provides long-term bonding. In additional embodiments, the long-term bonding agent is contained and released from within the closure member to provide easier application.

Various embodiments of the invention provide for a device for improving the process of wound closure in the form of a kit offering step-appropriate cleanliness or sterility and easier to use working surfaces, having an instruction layer available on opening the kit, with a durable card of wound care instructions to be given to the patient; a cleaning layer containing non-sterile gloves, syringe and splash prevention mechanism to help the health care provider clean the wound; and a wound closure layer containing closure components and kept separate until the wound area is prepared for closure.

In some embodiments, all or some layers are physically separated. In various embodiments, a sterile field layer may be included for wound closure techniques requiring sterility. In additional embodiments, layers subsequent to the sterile field layer are sterile.

In further embodiments, a mechanism exists for introducing fluid or other substance into a specific portion of the kit in advance of starting the procedure. In some embodiments, a portion of the outside of the kit is covered by a removable barrier, which, when opened, creates a window to a specific layer of the kit. In additional embodiments, the removable barrier creates a window to a section of one layer, which contains an independent tub or container to keep a substance or fluid separated from the rest of the layer and kit. In some embodiments, the container within the layer is waterproof and can be used for sterile water, local anesthetic, or other substance or medication.

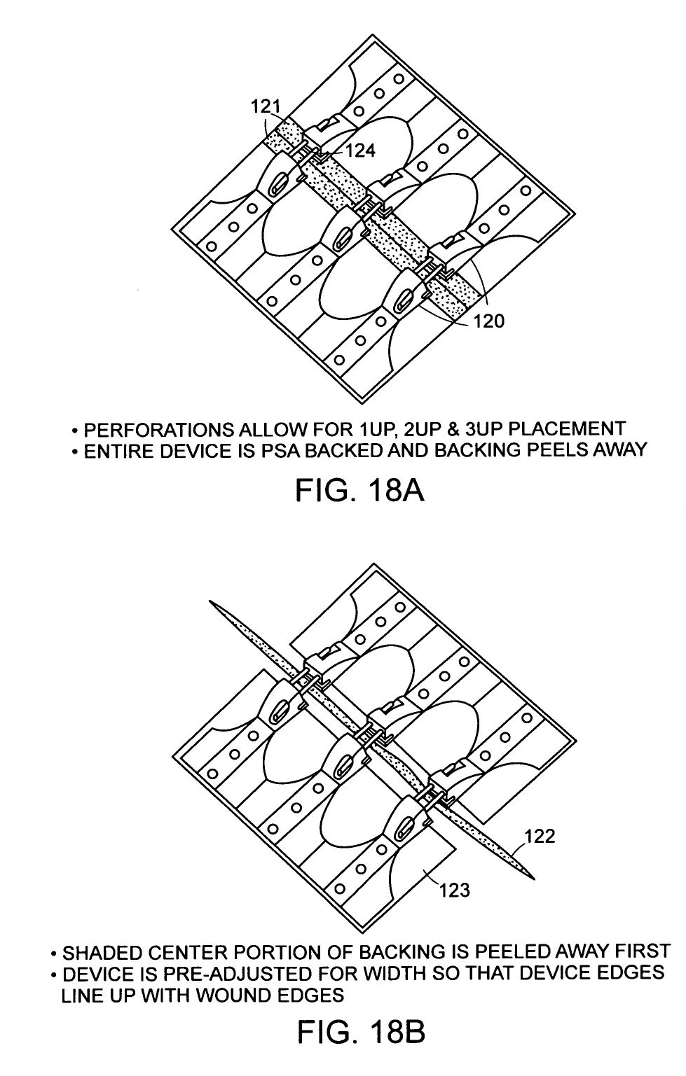

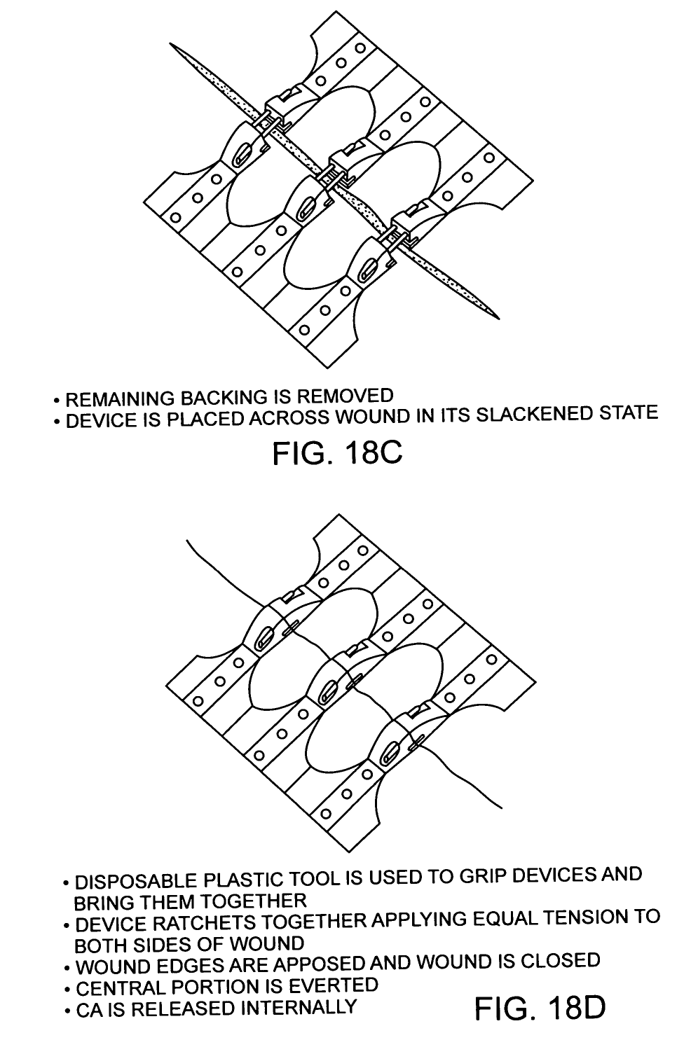

FIGS. 18A-18D show a wound closure device containing multiple closure components and a method of its use. In this embodiment, the doctor can use perforations to adjust the number of closure components 120 based upon wound length (such as, for example, one, two or three closure components) as shown in FIG.18A. The entire device in this embodiment is backed with a pressure sensitive adhesive and a backing that peels away. The backing for shaded center portion 121 is peeled away first. The device in this embodiment is pre-adjusted for width (which in some embodiments is facilitated using bridging element 124) so that the device edges line up with the wound edges when the device is placed over wound 122 such that one member of each closure component is on one side of wound 122 and the second member of each closure component is on the other side of wound 122 as shown in FIG. 18B. The remaining backing 123 is removed. FIG. 18C shows the device after the removal of the remaining backing. The device is placed across the wound as described above in its slackened state. Another device, such as for example a disposable plastic tool, can be used to grip the components and bring the two members of each component together. (In some embodiments, a doctor or health care provider can manually do this as well.) When this is done, the two members of each closure component ratchet together applying equal tension to both sides of the wound in this embodiment. When this is completed, the wound edges are apposed, and the wound is closed. This is shown in FIG. 18D. The edges of the wound, the central portion, are everted. In this embodiment, cyanoacrylate is released internally.

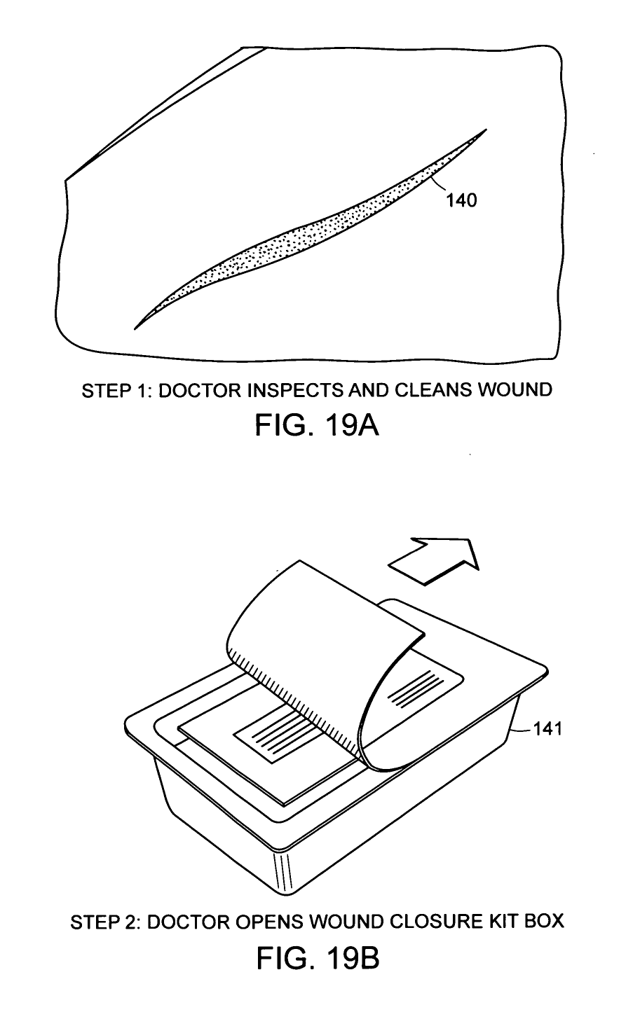

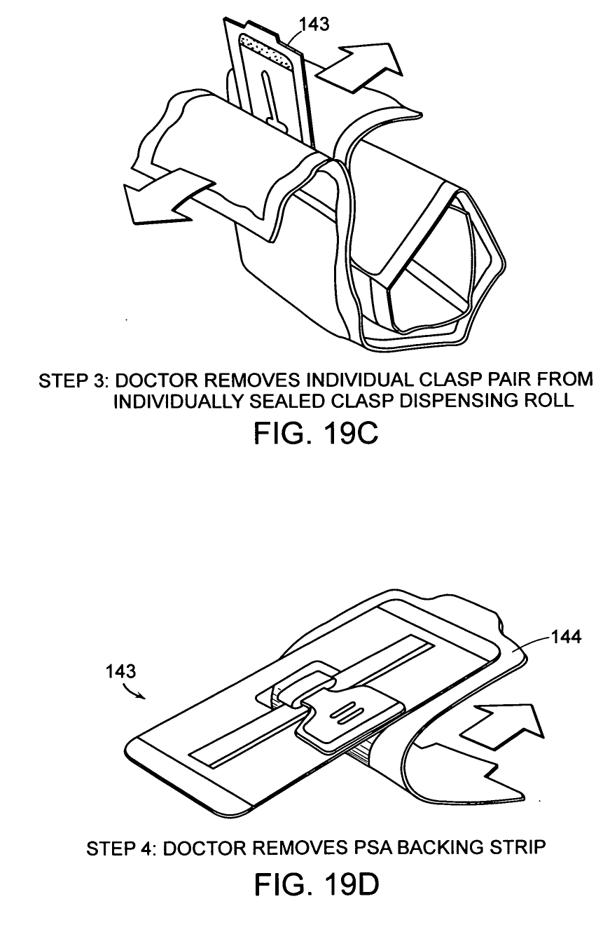

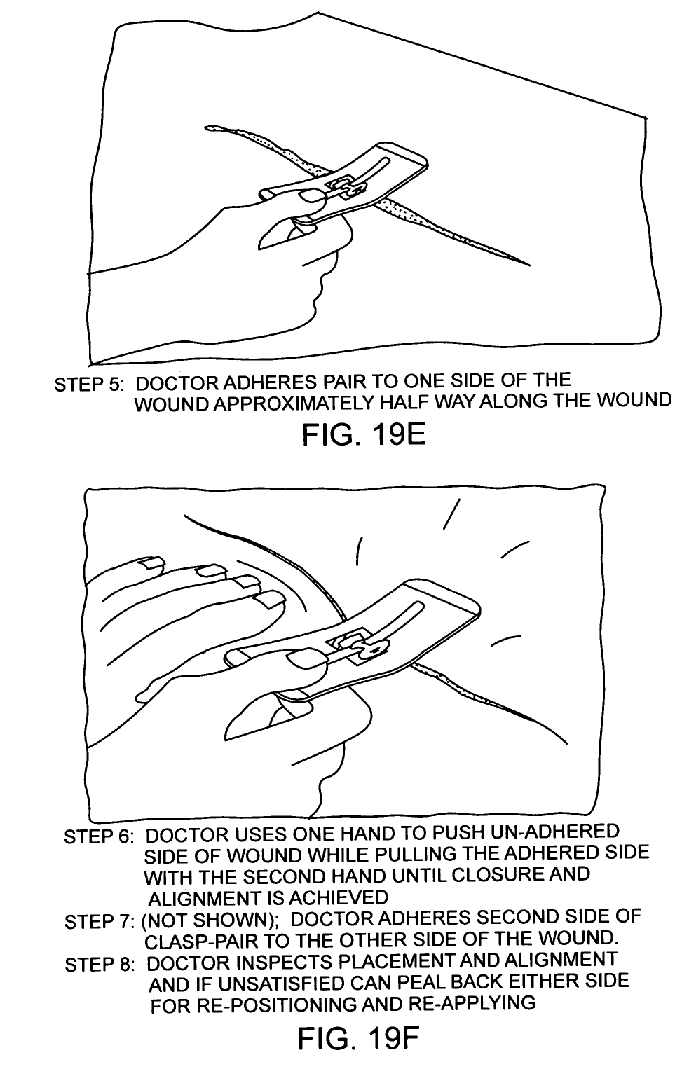

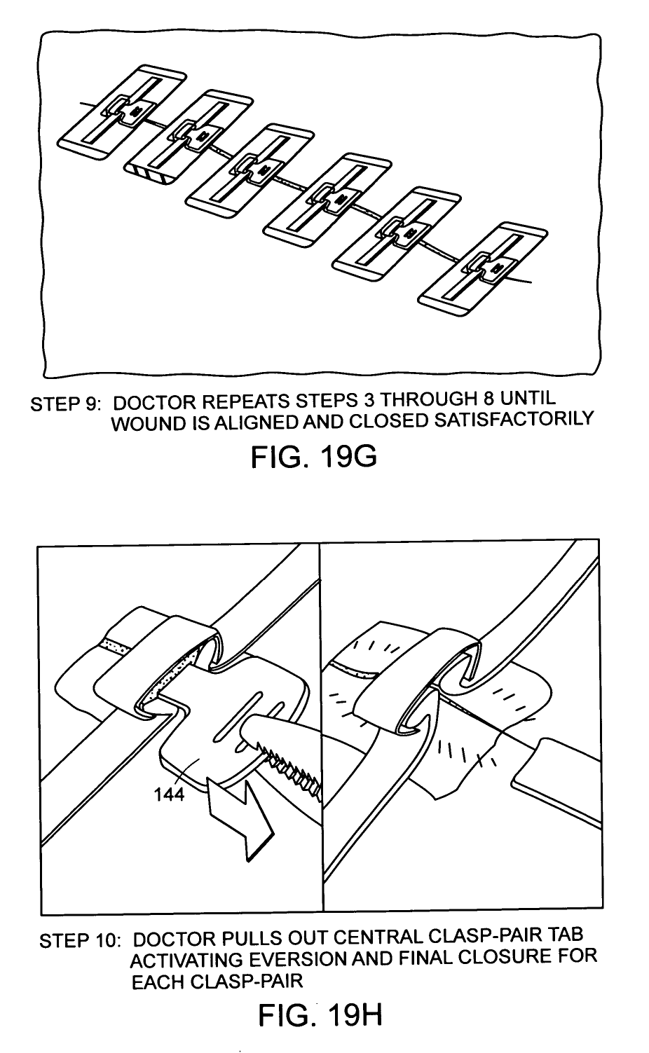

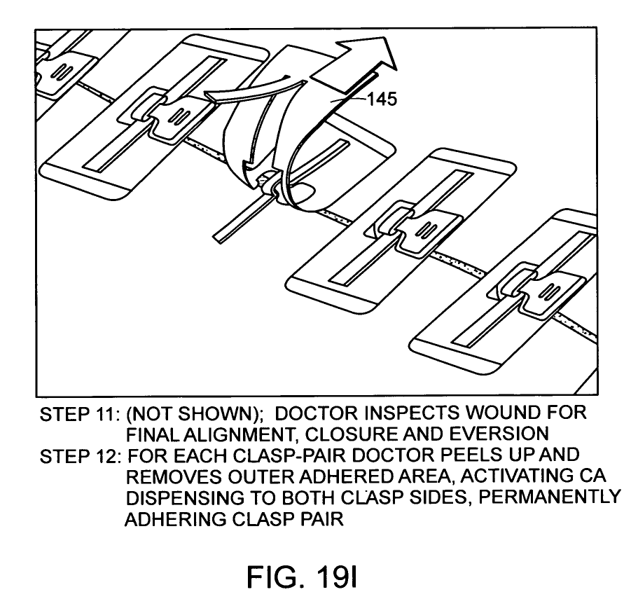

FIGS. 19A-19I show a method using a kit for a closure system including the kit, various components contained within the kit, and a method of its use. FIG. 19A shows wound 140. The doctor or healthcare provider inspects and cleans the wound. In FIG. 19B the doctor opens kit 141 for closing a tissue opening by removing a cover from the kit. In this embodiment, the kit is a wound closure kit box. As shown in FIG. 19C, individual closure component 143, such as for example a clasp pair in this embodiment, is removed from an individually sealed clasp-dispensing roll. The doctor removes pressure sensitive adhesive back strip 144 from closure component 143, as shown in FIG. 19D. FIG. 19E shows the doctor adhering the closure component to one side of the wound approximately halfway along the wound. The doctor can use one hand to push an unadhered side of the wound while pulling the adhered side with the second hand until closure and alignment of the wound is achieved as shown in FIG. 19F. The doctor then adheres the second side of the clasp-pair closure component to the other side of the wound and inspects placement and alignment of the closure component. If unsatisfied, the doctor can peel back either side of the closure component for repositioning and reapplying. These steps are repeated until the entire wound is aligned and closed satisfactorily as shown in FIG. 19G. FIG. 19H shows the doctor pulling out central clasp-pair tab 144 to activate eversion of the wound edge and final closure for each clasp-pair closure component. The doctor may then inspect the wound for final alignment, closure and eversion. Finally, as shown in FIG. 19I, the doctor peels up and removes an outer adhered area for each clasp pair closure component, which activates the dispensing of cyanoacrylate to both sides of the clasp, semi-permanently adhering the clasp-pair to the skin.

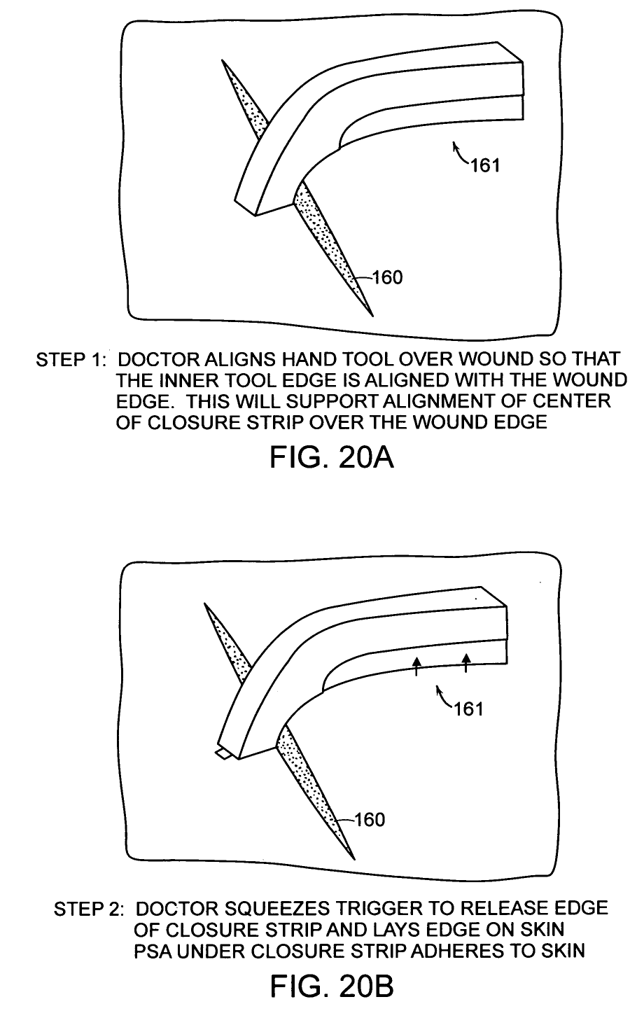

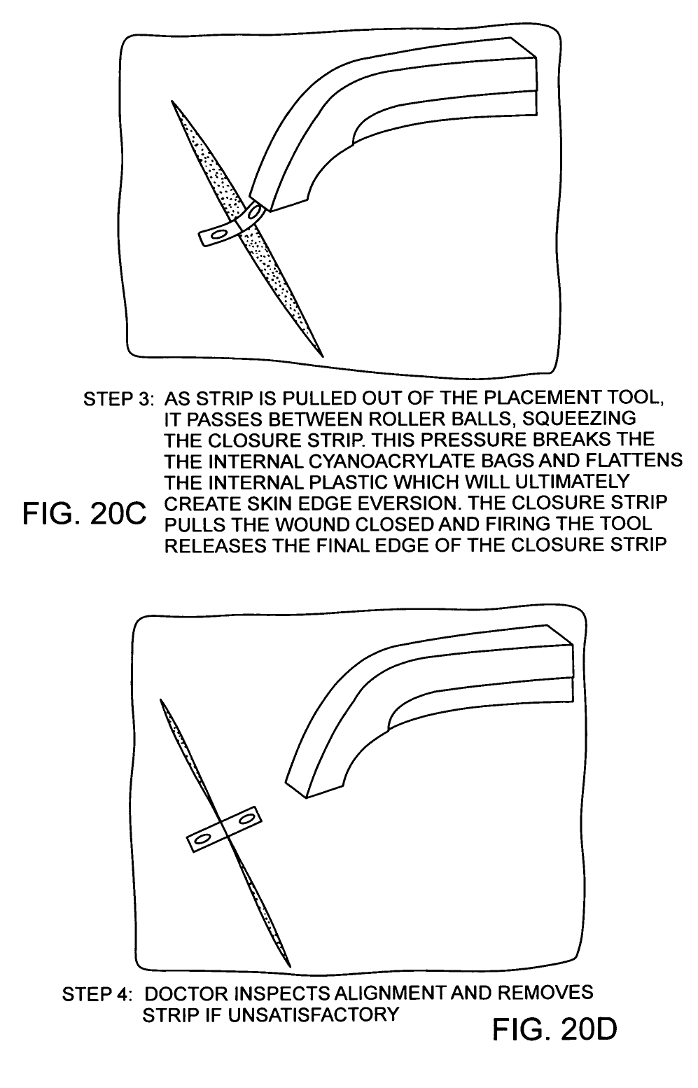

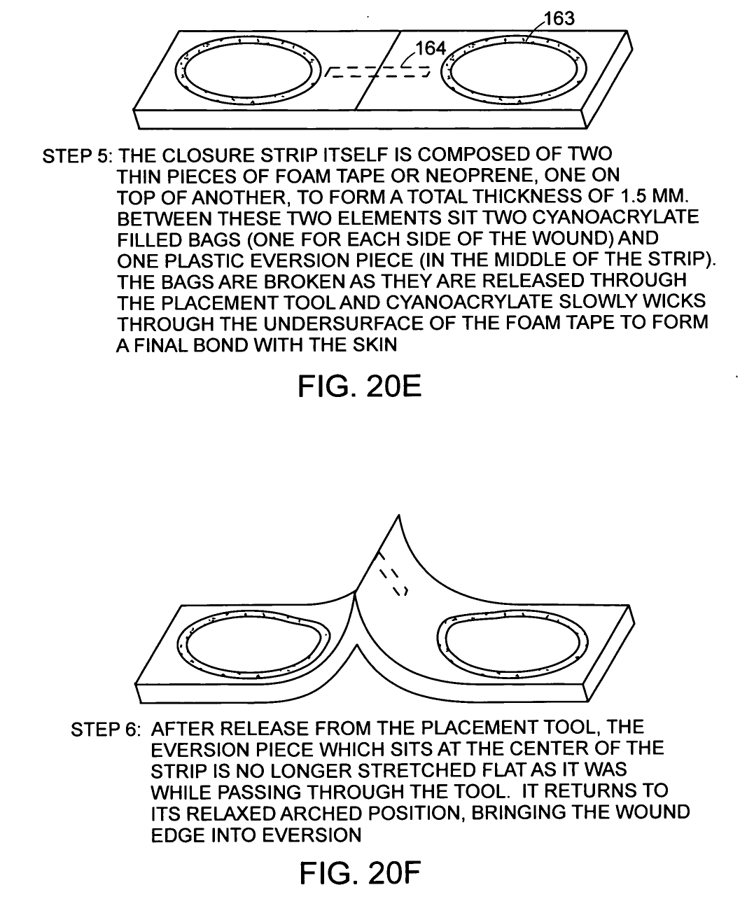

FIGS. 20A-20F show a wound closure device containing multiple pairs of wound closure components and a method of its use. In the embodiment depicted in these figures, the doctor uses hand tool 161, which is aligned with respect to wound 160 such that the inner edge of tool 161 is aligned with the wound edge as shown in FIG. 20A. This provides for alignment of the center of a closure strip over the wound edge. Thus, in this embodiment, the closure device is one piece, such as for example, a thick strip having a mechanism for mechanical eversion and a mechanism for internal release of a second adhesive. The doctor squeezes the trigger of hand tool 161 to release the edge of a closure strip and lays the edge on the skin as shown in FIG. 20B. A pressure sensitive adhesive under the closure strip adheres to the skin. In various embodiments, as the strip is pulled out of the placement tool, it passes between roller balls or another mechanism, squeezing the closure strip. This pressure breaks internal reservoirs containing a second adhesive, such as for example, cyanoacrylate bags in the closure strip, and flattens the internal plastic that will ultimately create eversion of the skin at the wound edge. The closure strip pulls the wound closed and a mechanism, for example, a firing tool, releases the final edge of the closure strip. In some embodiments, manual pressure or other mechanism may be used to facilitate wound closure. FIG. 20C shows the release of the closure strip from the hand tool. FIG. 20D shows the closure strip after it has been placed (deployed or applied). The doctor can then inspect the alignment and remove the strip if the placement or the wound closure is unsatisfactory. In some embodiments, the closure strip itself is composed of at least two layers, such as for example thin pieces of foam tape or neoprene, one on top of another. In one embodiment, the total thickness of the two layers is about 1.5 mm to about 3 mm. As shown in FIG. 20E, between these two layers are two reservoirs 163, such as for example bags, (one for each side of the wound), which in some embodiments are filled with an adhesive, such as for example, cyanoacrylate. Thus, one reservoir can be on each side of the tissue opening. In some embodiments, the reservoirs are about 0.5 mm thick when filled and do not add substantially to the overall thickness of the device. In other embodiments, more cyanoacrylate is used, and the total thickness of the device is about 2.5 mm to about 3 mm. Also between these two layers is a plastic eversion piece (eversion element) 164 in the middle of the strip. In various embodiments, these bags are broken as they are released through the placement tool, as discussed above, and cyanoacrylate slowly wicks through the undersurface of the foam tape to form a final bond securing the closure strip to the skin. After release from the placement tool, the eversion piece, which sits at the center of the strip, is no longer stretched flat as it was while passing through the tool. Thus, upon application (when placed or deployed), it returns to its relaxed arched position, bringing the wound edge into eversion as shown in FIG. 20F.

In some embodiments, the second layer is a porous material. In additional such embodiments, the porous material holds the second adhesive, such as for example, cyanoacrylate. Thus, the reservoirs for the second adhesive may not be present; however, such reservoirs could also be used in conjunction with the porous material. In some embodiments, the second adhesive is dispensed intentionally using pressure. In some embodiments, the second layer is sealed by a material that provides pressure-induced porosity. In additional embodiments, this layer is a selectively-permeable layer, which can control, among other things, the direction of flow of a material.

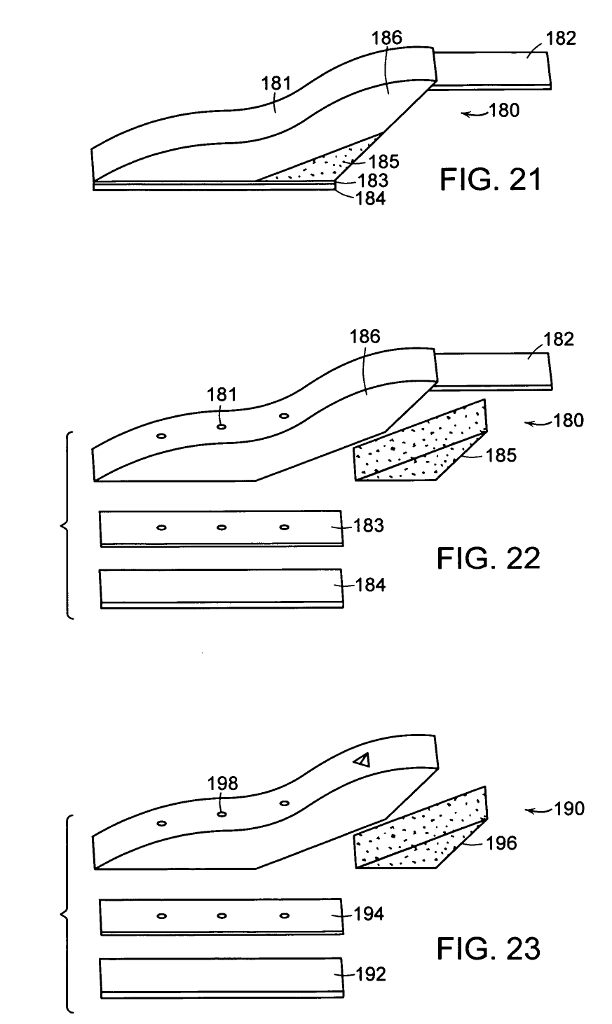

FIG. 21 is a schematic view of male member 180 of an embodiment. In this embodiment, through-holes 181 provide access to apply additional material, including but not limited to glue-dissolving agents.

FIG. 22 is an exploded view of FIG. 21. This exploded embodiment of the design for male member 180 shows sponge layer 183 that provides the adhesive dispensing mechanism to the lower pressure sensitive adhesive layer 184. Wedge 185 illustrates a hard material (such as, for example, plastic) that ensures edge eversion. Upper body 186 is comprised of a flexible (softer) material (such as, for example, a polymer) allowing flexibility to accommodate movement and swelling, while not sacrificing adhesion strength. FIGS. 21 and 22 illustrate an alternative male latching mechanism (connective element) 182 that allows fine adjustment to placement errors, such as, for example, to correct minor placement errors.

In some embodiments, second layer 183 is a porous material. In additional such embodiments, the porous material holds the second adhesive, such as for example, cyanoacrylate. In addition, reservoirs for the second adhesive could also be used in conjunction with the porous material. In some embodiments, the second adhesive is dispensed intentionally as the connective elements become engaged or after the connective elements are engaged in the latching step. In some embodiments, the second layer is sealed by a material that provides pressure-induced porosity. In additional embodiments, this layer is a selectively-permeable layer, which can control or regulate, among other things, the direction of flow of a material.

In some embodiments, one or more of the members have one or more holes through all but the bottom layer (such as for example the “through-holes” discussed above), which in some embodiments comprises the first adhesive. In various embodiments, such holes can be used for application of various materials, including but not limited to a dissolving agent, to the interior of the surface that is affixed to the skin. In additional embodiments, the hole, which forms a channel, is sealed against the flow of the second adhesive. This can prevent or at least minimize diffusion of materials introduced through the channel to other parts of the member. Sealing the channel against the flow of the second adhesive helps to maximize the effectiveness of applying other materials through this aperture.

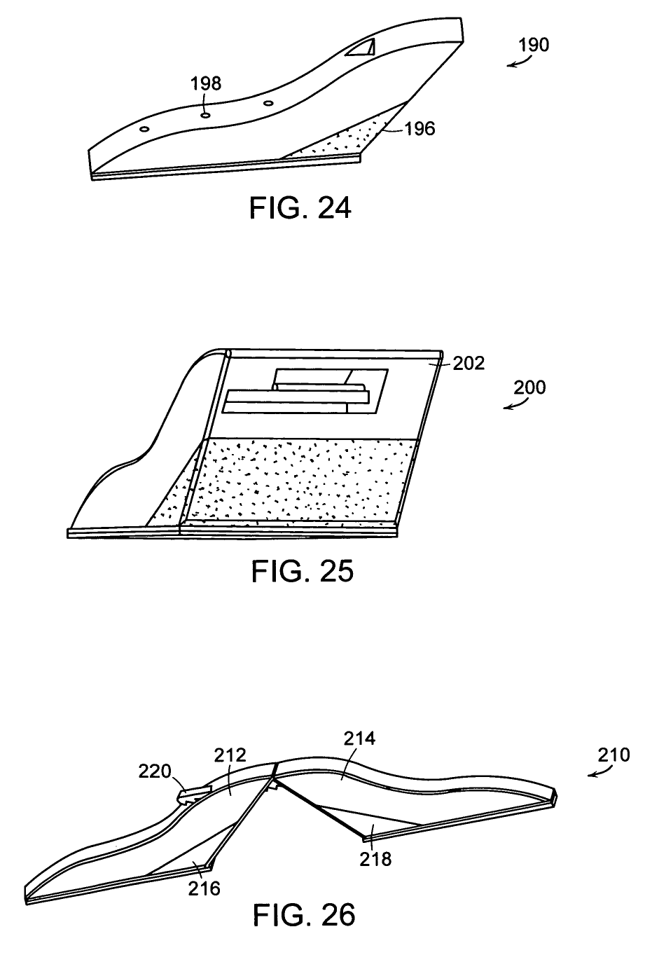

FIG. 23 is an exploded view of a female member 190 corresponding to the male member depicted in FIGS. 21 and 22. FIG. 24 is a schematic view of a female member corresponding to the male member depicted in FIGS. 21 and 22.

FIG. 25 is a schematic view of a female member 200, such as, for example, shown in FIGS. 23 and 24, showing a latching mechanism 202. In one such embodiment the latching mechanism 202 is provided by a cantilevered extrusion. FIG. 26 is a schematic view of a male member 214 and a female member 212 fully engaged. In the embodiment depicted in FIG. 26, the male and female members are engaged at the limit of the fine adjustment mechanism 220, which in some embodiments could be extended to allow additional space between the male and female member to facilitate adjusting for incorrect placement of either or both components.

Care after the closure procedure can be critical to wound healing outcome. For post-procedural care, a wound dressing system can be used. In some embodiments, dressings may be clearly labeled for use with reference to a specific time or period, such as, for example, time from initial injury or period of wound healing. A simple to follow regimen of dressing changes may be performed by the patient. By way of non-limiting example, within the first two days, as an initial skin covering forms, contemplated aspects of the dressing include moisture sealing and pain relief. During subsequent days of normal healing, as support below the skin is normally laid down, in some embodiments, contemplated aspects of the dressing include moisture sealing, infection sensing through pH, exudates reaction, or other factors, and transparency for ease of inspection. During later days of normal healing, as the wound matures, in various embodiments, contemplated aspects of the dressing include vitamin E and other scar reducing compounds.

In various embodiments, the invention provides for a system for promoting wound healing after wound closure having a set of different dressings targeted to time-defined periods of wound healing and a series of patient controlled steps to care for the wound, including placement of specific dressings at specified times. In some embodiments, the dressings are targeted to times corresponding to the physiologic phases of wound healing. In other embodiments, one or more of the dressings is moisture impermeable for use when a moist environment promotes healing. In further embodiments, one or more of the dressings is transparent to allow visualization of a potential infection. In additional embodiments, one or more of the dressings contain a chemical indicator triggered by physiologic changes associated with infection. In some embodiments, the indicator reacts to specific pH levels or protein exudates. In further embodiments, one or more of the dressings releases vitamin E to reduce scarring. In some embodiments, the patient is given a compound or compounds to regularly apply to the dressing to reduce scarring. In additional embodiments, one or more of the dressings releases a chemical solvent leading to easy removal of an adherent wound closure device.

In view of the wide variety of embodiments to which the principles of the present invention can be applied, it should be understood that the illustrated embodiments are exemplary only, and should not be taken as limiting the scope of the present invention. For example, the steps of the flow diagrams may be taken in sequences other than those described, and more or fewer components may be used in the diagrams.

Other aspects, modifications, and embodiments are within the scope of the following claims.

1. A closure device for closing a tissue opening, comprising:
one or more closure components, each closure component comprising a first member and a second member;
each of the first and second members having a first surface that adheres to a tissue surface proximate to the tissue opening, the first surface having at least one adhesive;
each of the first and second members having a second surface substantially orthogonal to the first surface and having at least one connective element on the second surface; and
each of the first and second members having a transitional region between the first surface and the second surface which is contoured to evert an edge of the tissue opening upon the drawing together of the first and second members by the engagement of the at least one connective element.

2. The closure device of claim 1, wherein the connective element is a locking mechanism.

3. The closure device of claim 2, wherein the locking mechanism comprises a locking member extending from the second surface of the first member and the second surface of the second member having a receiving member.

4. The closure device of claim 2, wherein each of second surfaces have at least one locking member and at least one receiving member.

5. The closure device of claim 4, wherein the first member and the second member are identical.

6. The closure device of claim 1, wherein the connective elements comprise a ball and socket mechanism.

7. The closure device of claim 1, wherein the connective elements comprise a rachet mechanism.

8. The closure device of claim 1, wherein the connective elements comprise a suture ligature.

9. The closure device of claim 1, wherein the connective elements comprise a lock and key mechanism.

10. (canceled)

11. The closure device of claim 1, wherein the connective elements comprise a photobonded mechanism.

12. (canceled)

13. The closure device of claim 1, wherein the connective element of the first member engages the connective element of the second member when the second surface of the first member is in operative relation to the second surface of the second member.

14. The closure device of claim 13, wherein the engaged connective elements provide a vertical force orthogonal to a plane of the tissue opening on at least one edge of the tissue opening.

15. The closure device of claim 13, wherein the process of engaging the connective elements brings edges of the tissue opening together and everts the edges of the tissue opening.

16. The closure device of claim 1, wherein the first and second members are releasably coupled.

17. The closure device of claim 1, further comprising a closure release mechanism.

18. (canceled)

19. The closure device of claim 1, wherein the connective element is an adhesive.

20. The closure device of claim 19, wherein the adhesive is glue.

21. The closure device of claim 1, further comprising a mechanism to adjust the placement of the members.

22. The closure device of claim 21, wherein the mechanism to adjust the placement of the members is a sliding track.

23. The closure device of claim 21, wherein the mechanism to adjust the placement of the members is the adhesive on the first surface providing for repositioning of the member.

24. The closure device of claim 21, wherein the mechanism to adjust the placement of the members is a hinge.

25-28. (canceled)

29. The closure device of claim 1, further comprising a sliding lock to position and hold the first and second members in proximity to each other.

30. The closure device of claim 1, further comprising protrusions extending from the member to facilitate handling of the components.

31. (canceled)

32. The closure device of claim 1, wherein the members are plastic.

33. The closure device of claim 1, wherein the members are metal.

34. The closure device of claim 1, wherein the members are bioabsorbable.

35. The closure device of claim 1, wherein at least one of the members is flexible.

36-38. (canceled)

39. The closure device of claim 1, wherein the adhesive is a semi-permanent skin bonding agent.

40. The closure device of claim 39, wherein the semi-permanent skin bonding agent is a pressure sensitive adhesive.

41. The closure device of claim 39, wherein the semi-permanent skin bonding agent is a skin glue.

42. The closure device of claim 1, further comprising a second adhesive.

43. The closure device of claim 1, wherein at least one member comprises an opening for application of a second adhesive provided in a plane that is substantially parallel to the first surface and spaced apart from the first surface, the opening extending through to the first surface.

44-46. (canceled)

47. The closure device of claim 1, wherein each member comprises two or more layers.

48. The closure device of claim 47, wherein at least one layer is porous.

49. The closure device of claim 48, wherein the porous layer is sealed by a material that provides pressure-induced porosity.

50. The closure device of claim 48, wherein the second adhesive is present in the porous layer.

51. The closure device of claim 1, wherein the transitional region is tapered.

52. The closure device of claim 1, wherein the transitional region is beveled.

53. The closure device of claim 1, wherein the transitional region is arcuate.

54. The closure device of claim 1, wherein the transitional region is chamfered.

55. The closure device of claim 1, wherein the transitional region is sloped.

56-106. (canceled)

107. A closure system for a tissue opening, comprising:
a temporary closure device comprising a deformable perimeter defining an opening, the deformable perimeter having a first surface and at least one adhesive on the first surface, and wherein the temporary closure device surrounds the tissue opening and is sufficiently compliant to deform and exert pressure on opposing sides of a tissue opening and is sufficiently rigid to maintain the deformed shape and maintain the pressure on opposing sides of the tissue opening and wherein opposing sides of the perimeter are pressed together exerting a force on tissue underneath the temporary closure device to bring edges of a tissue opening closer together;
one or more closure components, each closure component comprising a first member and a second member;
each of the first and second members having a first surface that adheres to a tissue surface proximate to the tissue opening, the first surface having at least one adhesive;
each of the first and second members having a second surface substantially orthogonal to the first surface and having at least one connective element on the second surface;
each of the first and second members having a transitional region between the first surface and the second surface which is contoured to evert an edge of the tissue opening upon the drawing together of the first and second members by the engagement of the connective elements; and
an applicator device for applying the closure components, the applicator device comprising at least one of a mechanism to hold the closure components, a mechanism to release the closure components, and a mechanism to affix the closure components.

108-120. (canceled)

121. The closure system of claim 107, further comprising an applicator device for applying the closure components, the applicator device comprising at least one of a mechanism to hold the closure components, a mechanism to release the closure components, and a mechanism to affix the closure components.

122-125. (canceled)

126. The closure system of claim 121, wherein the applicator device further comprises a mechanism to adjust placement of the closure components.

127. (canceled)

128. The closure system of claim 121, wherein the applicator device further comprises a mechanism for securing the closure components.

129-130. (canceled)

131. A method for closing a tissue opening, comprising:
positioning a closure device for closing a tissue opening, comprising one or more closure components, each closure component comprising a first member and a second member, each of the first and second members having a first surface that adheres to a tissue surface proximate to the tissue opening, the first surface having at least one adhesive, each of the first and second members having a second surface substantially orthogonal to the first surface and having at least one connective element on the second surface, and each of the first and second members having a transitional region between the first surface and the second surface which is contoured to evert an edge of the tissue opening upon the drawing together of the first and second members by the engagement of the connective elements;
drawing the first and second members into proximity such that the connective element on the second surface of the first member engages the connective element on the second surface of the second member; and
securing each of the first and second members to a tissue surface.

132. The method of claim 131, wherein the closure members are positioned adjacent to an edge of the tissue opening.

133. The method of claim 131, wherein the transitional region is tapered.

134. The method of claim 131, wherein edges of the tissue opening adhere to the transitional region and are everted.

135. The method of claim 131, wherein bringing the first and second members into proximity such that the connective element on the second surface of the first member engages the connective element on the second surface of the second member everts the wound edge.

136. The method of claim 131, wherein the transitional region is beveled and drawing the first and second members into proximity such that the connective element on the second surface of the first member engages the connective element on the second surface of the second member brings the beveled transition region of the first member into proximity with the beveled transition region of the second member and everts the wound edge.

137. The method of claim 131, further comprising adjusting the position of at least one of the closure members after positioning it.

138. (canceled)

139. (canceled)

140. The method of claim 131, wherein the first and second members are secured with an adhesive.

141. The method of claim 140, wherein the adhesive is cyanoacrylate.

142. The method of claim 140, wherein the adhesive is released from at least one member.

143. (canceled)

144. The method of claim 140, wherein each member comprises two or more layers, at least one layer being porous, and the porous layer being sealed by a material that provides pressure-induced porosity.

145. (canceled)

146. The method of claim 131, wherein the two or more closure components are positioned in spaced relationship.

147. The method of claim 131, further comprising affixing the positioned members.

148. The method of claim 147, wherein the members are affixed with an adhesive.

149. The method of claim 148, wherein the adhesive is a pressure sensitive adhesive.

150. The method of claim 148, wherein the adhesive is glue.

151. The method of claim 131, further comprising bringing the edges of the tissue opening into proximity prior to positioning the closure members.

152-164. (canceled)
